(19) United States
(12) Patent Application Publication
(10) Pub. No.: US 2024/0207282 A1
(43) Pub. Date: Jun. 27, 2024

(54) METHOD OF INCREASING CHAPERONE MEDIATED AUTOPHAGY BY STABILIZING THE INTERACTION OF RETINOIC ACID RECEPTOR-ALPHA AND AN INHIBITOR

(71) Applicant: ALBERT EINSTEIN COLLEGE OF MEDICINE, Bronx, NY (US)

(72) Inventors: Ana Maria CUERVO, Bronx, NY (US); Evripidis GAVATHIOTIS, Flushing, NY (US)

(21) Appl. No.: 18/556,419

(22) PCT Filed: Apr. 21, 2022
(86) PCT No.: PCT/US2022/025753

§ 371 Date: Oct. 20, 2023

Related U.S. Application Data
(60) Provisional application No. 63/177,674, filed on Apr. 21, 2021.

(51) Int. Cl. A61K 31/538; A61K 31/498; A61K 47/54; A61P 27/02; G01N 33/68

(52) U.S. Cl. CPC A61K 31/538; A61K 31/498; A61K 47/545; A61P 27/02; G01N 33/6896; G01N 2800/2821

(57) ABSTRACT

This disclosure provides a method of stabilizing the interaction of a Retinoic Acid Receptor-alpha (RARα) and a corepressor, Nuclear Receptor Corepressor 1 (NCoR1) by contacting the RARa with an amount of a Chaperone Mediated Autophagy (CMA) Activator sufficient to stabilize the RARα-NCoR1 interaction. Stabilizing the RARα/corepressor interaction can prevent a neurodegenerative disorder in a subject at risk for developing the neurodegenerative disorder or slow the advancement of a neurodegenerative disorder in a subject having an early symptom or biomarker of the neurodegenerative disorder. The disclosure also provides a method of maintaining preventing or slowing the advancement of a retinal degenerative disorder in a subject having an early symptom or biomarker of the retinal degenerative disorder. The neurodegenerative disorder can be Alzheimer's disease (AD), Lewy body dementia, Parkinson's disease (PD), Huntington's disease, Amyotrophic lateralsclerosis (ALS), Frontotemporal dementia (FTD), Spinocerebellar ataxias (SCAs). The retinal degenerative disorder can be retinitis pigmentosa.

### CROSS REFERENCE TO RELATED APPLICATION

The application claims priority to U.S. Provisional Appl. No. 63/177,674, filed Apr. 21, 2021, which is hereby incorporated by reference in its entirety.

### BACKGROUND

Loss of proteostasis underlies the basis of multiple age-related degenerative disorders. Chaperone-mediated autophagy (CMA) activity, essential in the cellular defense against proteotoxicity, declines with age.

Maintenance of proteostasis is essential for normal cellular function and for adaptation to the always changing extracellular environment. Chaperones and the proteolytic systems are the major components of the proteostasis network. Gradual loss in functionality of some of these proteostasis pathways with age has been proposed to accelerate the course of degenerative conditions that afflict the elderly. We have previously shown that chaperone-mediated autophagy (CMA), a selective mechanism for degradation of cytosolic proteins in lysosomes, declines with age in most tissues from rodents and humans. Furthermore, CMA is vulnerable to the toxic effect of pathogenic proteins that accumulate in neurodegenerative diseases such as Parkinson's disease or tauopathies. Inhibition of CMA in these conditions further contributes to proteotoxicity in the affected tissues and perpetuates the proteostasis failure.

Lower CMA activity in aging mainly results from reduced levels of the lysosome-associated membrane protein type 2A, LAMP2A (L2A), the receptor for the substrate proteins delivered to lysosomes by the chaperone Hsc70. Binding of the substrate to the receptor, the limiting step in CMA, triggers L2A assembly into a multimeric translocation complex used by the substrates to reach the lysosomal lumen for degradation. Preventing the decline of CMA in old rodents through genetic manipulations (L2A overexpression) has proven effective in maintaining organ function. Genetic L2A upregulation is also protective in models of Parkinson's disease-related neuronal toxicity. Thus, CMA activation may be beneficial in diseases where its inhibition has a pathogenic role. CMA has proven to be central to proteostasis maintenance in the retina and to become the main defense against proteotoxic insults with aging, as the other types of autophagy start to fail

In previous studies, we identified that CMA is under the negative regulation of signaling through the retinoic acid receptor alpha (RARα) and developed first-in-class small molecules capable of upregulating CMA in vitro by blocking the RARα-mediated inhibition on this type of autophagy. Common inhibitors and antagonists of RARα, although also effective in activating CMA, have a negative impact on other types of autophagy such as macroautophagy, since RARα is an activator of this pathway. There exists a need for small molecules that selectively activate CMA without affecting other forms of autophagy. This disclosure fulfills that need and provides additional advantages.

### SUMMARY

This disclosure provides a method of stabilizing the interaction of a Retinoic Acid Receptor-alpha (RARα) and a corepressor, Nuclear Receptor Corepressor 1 (NCoR1) comprising contacting the RARα with an amount of a Chaperone Mediated Autophagy (CMA) Activator sufficient to stabilize the RARα-NCoR1 interaction.

In an embodiment, this disclosure provides method of preventing or slowing the advancement of a neurodegenerative disorder in a subject having an early symptom or biomarker of the neurodegenerative disorder, comprising administering an amount of a CMA activator sufficient to stabilize the interaction of RARα and the corepressor NCoR1 in vivo.

The disclosure further provides a method of maintaining preventing or slowing the advancement of a retinal degenerative disorder in a subject having an early symptom or biomarker of the retinal degenerative disorder, comprising administering to the subject an amount of a CMA activator sufficient to stabilize the interaction of RARα and the corepressor NCoR1 in the subject's retina.

The disclosure also provides a method of maintaining proteostasis in the retina of a subject comprising administering an amount of a CMA activator to the subject sufficient to achieve a concentration of the CMA activator in the subject's retina sufficient to stabilize an interaction between RARα and NCoR1 in vitro.

The disclosure further provides a method of increasing Lamp 2A levels in neurons or retina of a subject in need of treatment for an age-related neurodegenerative disorder or retinal degenerative disorder, comprising administering to the subject an amount of a CMA activator sufficient to stabilize the interaction of RARα and the corepressor NCoR1 in the subject's retina or neurons.

In certain embodiments the CMA Activator is an Activator capable of hydrogen bonding with Thr 233 in the RARα.

The CMA Activator can also be an Activator capable of hydrophobic interaction with at least one of the following RARα (human consensus sequence) amino acids: Pro 407, Leu 409, Ile410, Pro408, and Ile 236 and/or with at least one of the following RARα (human consensus sequence) amino acids: Leu 266, Ile270, Phe302, and Leu305.

### BRIEF DESCRIPTION OF THE DRAWINGS

FIG. 1. CA39 and CA77 activate CMA in a dose-dependent manner. FIG. 1A. Molecular docking of CA39 (left) and CA77 (right) in the binding pocket of inactive RARα. FIG. 1B. A close view of the binding pose of AR7 in the binding pock of inactive RARα in ribbon highlighting RARα interacting residues in sticks. AR7 occupies a hydrophobic pocket present in the inactive RARα formed by helices h3, h10, and h12. FIG. 1C. Predicted RARα amino acid interactions with CA39 and CA77. FIG. 1D. Quantification of CMA activity in NIH3T3 cells stably expressing the KFERQ-PS-Dendra after addition of increasing concentrations of CA39 and CA77 for 12 (left) or 24 (right) hours. n>2,500 cells in 4 independent experiments. FIG. 1E Quantification of CMA activity in the same cells after addition of increasing concentrations of the compounds for 12, 24 h and 12 h after washing (w) them out from the media. n>1,500 cells in 3 independent experiments. All values are mean+s.e.m. One-way Anova (D, E) followed by Bonferronis's multiple comparisons post-hot test were used. Significant differences with untreated samples are indicated in e and among the different incubation protocols in f. **p<0.01, ***p<0.001, ****p<0.0001. ns: no significant difference.

FIG. 2. Lack of effect of CA39 and CA77 on macroautophagy activity. FIG. 2A. Proteolysis of long half-life intracellular proteins was measured upon metabolic labeling for 48 h with 3H-leucine as described under methods in NIH3T3 without additions (None) or cultured in the presence of 10 μM CA39 or CA77 for the indicated times. n=3 independent experiments with triplicate wells. FIG. 2B. Immunoblot for LC3 in NIH3T3 incubated with 20 μM CA39 or CA77 for 16 h. Where indicated, lysosomal protease inhibitors (PI) were added to the incubation media 6 h before the end of the experiment. Representative immunoblot (left) and quantification of the rate of degradation of LC3-II (flux) relative to that in untreated (None) cells (right). n=4 independent experiments. FIG. 2C. Quantification of macroautophagy activity in NIH3T3 cells stably expressing the mCherry-GFP-LC3 reporter and treated with increasing concentrations of CA39, CA77 or the macroautophagy activator rapamycin for 16 h. Quantification of the amount of mCherry⁺ puncta (autophagic vacuoles, AV), mCherry⁺GFP⁺ puncta (autophagosomes, APG) and mCherry⁺GFP⁻ puncta (autolysosomes, AUT). n>1,100 in 3 different experiments. All values are mean+s.e.m. One-way Anova (FIG. 2B) or two-way Anova (FIG. 2A, C) followed by Bonferronis's multiple comparisons post-hot test were used. Significant differences with untreated samples are indicated in A, C and with the drugs in the legend in A. **p<0.01 and ****p<0.0001. ns: no significant difference.

FIGURE. 3. CA compounds induce a discrete transcriptional effect by promoting the interaction of RARα and NCoR1. FIG. 3A. (top) AR7-induced changes in expression of the indicated components of the CMA network. (bottom) Scheme shows the CMA network with changing components highlighted in bold. Values are expressed relative to untreated cells. n=3 different experiments. (modified from Kircher, P., et al., PLOS Biol. (2019) 18: e3000301) Values are expressed relative to untreated cells. n=3 different experiments. FIG. 3B. AR7-induced changes in expression of the indicated components of the CLEAR network (macroautophagy and lysosomal examples shown). All values are mean+s.e.m. One sample t and Wilcoxon test was used in FIG. 3A. FIG. 3C. Venn diagram of the additional genes showing significant (p<0.01) changes in expression in cells treated with the indicated compounds. FIG. 3D. Molecular docking of CA39 and CA77 is compatible within the inactive (left) conformation of RARα bound to NCoR1 peptide. RARα active conformation is shown for comparison. ATRA binds only to the active conformation. Hypothetical binding poses of CA39 (orange) and CA77 are not compatible within the active conformation of RARα due to steric clash (black dashed circle) and are only shown for clarity. FIG. 3E EC50 (μM) in fluorescence polarization assays with RARα and the NCoR1 peptide incubated without additions (no ligand) or in the presence of 10 μM of BMS614, CA39 and CA77. FIG. 3F. Immunoblot for NCoR1 and RARα of streptavidin pulldowns (top) or total cellular lysates (bottom) of NIH3T3 incubated without additions or in the presence of CA39 (10 μM) or biotin-CA (10 μM) for 24 h. IP: immunoprecipitation. This experiment was repeated 3 times. FIG. 3G CMA activity in NIH3T3 cells control (transduced with the lentiviral empty vector) or knock-down (KD) for NCoR1 (transduced with lentiviral carrying shRNA) incubated without additions (none) or in the presence of 20 μM CA39 or CA77 for 24 h. Left: representative images. Nuclei are highlighted with DAPI. Inserts show higher magnification. Right: Quantification of the number of puncta per cell. n>2,500 in 3 different experiments. Inset shows immunoblot for NCoR1 in controls and KD cells. All values are mean+s.e.m. One sample t and Wilcoxon test was used in FIG. 3A and FIG. 3C and two-way Anova followed by Bonferronis's multiple comparisons post-hot test in FIG. 3G. Starts in 3G show significant differences with untreated. The genotype effect is depicted in the inset. *p<0.05, **p<0.01, ***p<0.001 and ****p<0.0001.

FIGURE. 4. Transcriptional changes induced by CA compounds. FIG. 4A Fraction of genes significantly (p<0.01) upregulated or downregulated upon treatment with AR7, CA39 and CA77. FIG. 4B Types of transcripts with modified expression upon treatment with AR7, CA39 and CA77. FIG. 4C. Proteins coded by the genes significantly changed upon treatment with AR7, CA39 and CA77. Bottom shows their clustering upon gene set enrichment and node expansion analysis (using STRING database) including proteins added through node expansion. FIG. 4D. Functional groups of the nodes assigned to the 11 proteins with expression changed upon treatment with AR7, CA39 and CA77.

FIG. 5. In vitro and in silico ADME of CA compounds. FIG. 5A. In vitro solubility, metabolic stability (in liver microsomes from the indicated species) and permeability evaluation of CA39 and CA77, see Methods for experimental conditions. Comments on properties were added by an observer blinded to the nature of the compounds and the study. FIG. 5B. In silico QikProp (Schrodinger, LLC) analysis and ADME predictions for CA39 (left) and CA77 (right).

FIG. 6. CA compounds activate CMA in multiple tissues in vivo. FIG. 6A, B. Levels of CA39 and CA77 at the indicated times in plasma (6A) or brain (6B) after p.o. (oral, 30 mg/kg bw) and i.v. (intravenous, 1 mg/kg bw) administration in mice. n=3 mice per time point. FIG. 6C. Direct fluorescence in CD4+ T cells isolated from blood from KFERQ-Dendra mice i.p. injected daily with (30 mg/kg bw) CA39 for three consecutive days. Nuclei are highlighted in blue by Dapi. Right: higher magnification images. Arrows: puncta. FIG. 6D. Percentage of CD4+ T cells with CMA puncta>3 per cell (CMA+). n=5 mice per condition. FIG. 6E. mRNA levels of LAMP-2A (L2A) in CD4+ T cells activated for 24 h in the presence of CA39 and CA77 (10 μM). Values are expressed relative to no treated cells (None) after normalization by the housekeeping gene actin. Biological triplicates from 2 independent experiments. FIG. 6F. Representative images of livers from KFERQ-Dendra mice i.p. injected with CA39 and CA77 as in FIG. 6C. Nuclei are highlighted in blue by Dapi. Insets: higher magnification of sections. Arrows: puncta. FIG. 6G. Quantification of the average number of puncta per cell in liver. n=12 sections from 4 different mice. FIG. 6H Representative images of midbrain from KFERQ-Dendra mice i.p. injected with CA39 and CA77 as in FIG. 6C. Nuclei are highlighted in blue by Dapi. Insets: higher magnification of sections co-stained with MAPK2. Arrows: puncta. FIG. 6I. mRNA levels of LAMP-2A (L2A) in the same brain regions as in FIG. 6H. n=4 mice per condition. FIG. 6J. Representative images of flat mounted retinas from KFERQ-Dendra mice treated as in FIG. 6C. Nuclei are highlighted with DAPI. Bottom: Boxed areas at higher magnification. Insets: higher magnification images of. Right: Quantification of the number of Dendra⁺ puncta per cell. n=14 fields from 2 independent experiments. All values are mean+s.e.m. Two-way Anova followed by Sidak's multiple comparisons post-hoc test was used in FIG. 6A, B, unpaired two tailed t test was used in FIG. 6D and FIG. 6G, one sample t and Wilcoxon test in 6E and one-way Anova followed by Bonferroni's multiple comparisons post-hoc test in FIGS. 6I and 6K. *p<0.05, **p<0.01 and ***p<0.001 and ****p<0.001. ns: not significative.

FIG. 7. CA compounds have favorable blood brain barrier penetration and pharmacokinetics. FIG. 7A, B. Pharmacokinetics (PK) parameters of CA39 and CA77 in plasma (7A) and brain (7B) after p.o. (oral, 30 mg/kg bw) and i.v. (intravenous, 1 mg/kg bw) administration in mice. FIG. 7C. Brain to plasma (B/P) ratio of CA39 and CA77 at the indicated times after administration by i.v. or p.o. as in FIG. 7A. n=3 mice per time point. All values are mean+s.e.m. Two-way Anova followed by Sidak's multiple comparisons post-hoc test was used in FIG. 7C *p<0.05, **p<0.01 and ***p<0.001. ns: not significant.

FIG. 8. Toxicity assessment of the CA compounds in vivo. FIG. 8A. Mouse blood count at the end of a 5 months daily oral administration of vehicle (Veh) or a CA77 structure analogue (CA77d). Values are mean+s.e.m. from n=6 mice in each group. FIG. 8B-D. Representative images of sections of H&E stained liver (b), kidney (c) and lung (d) sections from the same mice. n=5 mice All values are mean+s.e.m. Two-way Anova followed by Bonferroni's multiple comparisons post-hot test and unpaired t test were both applied on A and no statistical differences were noted between vehicle and CA treated mice. FIG. 8E-G. Pathology scoring of the identified features in the three organs. Clinical relevant (CR) values are depicted as reference. Heat map of the individual features per organ (FIG. 8E), average scoring of all the features in each organ (FIG. 8F) or average of histological features per animal (FIG. 8G) are shown. n=3 mice per group. All values are mean+s.e.m. Two-way Anova (FIG. 8F) or one-way Anova (FIG. 8G) followed by Tukeys' multiple comparisons post-hot test were used. ***p<0.001 and ****p<0.0001. ns: no significant difference.

FIG. 9. CA compounds are cytoprotective in cells and tissues. FIG. 9A. Cell viability of NIH3T3 exposed to the indicated concentrations of paraquat (PQ) after 12 h treatment with 2 μM (left) or 10 μM (right) the indicated compounds. The treatment with PQ alone (None) or in the presence of the compounds lasted 12 h. n=3 independent experiments. FIG. 9B. Cell viability of NIH3T3 simultaneously exposed to the indicated concentrations of paraquat (PQ) and 2 μM (left) or 10 μM (right) of the indicated compounds for 12 h. n=3 independent experiments. FIG. 9C. Immunostaining for the indicated markers of rods and cones in whole mount retinas from rd10 mice maintained without additions (right eye; Vehicle) or in the presence of CA77 (left eye) for 24 h. Right: Quantification of the average area stained for rod arrestin (left) or number of opsin-positive counts per field (right) in retinas from the right and left eye of each animal. n=8 mice (representative experiment from 3 independent experiments). All values are mean+s.e.m. Two-way Anova followed by Bonferronis's multiple comparisons post-hot test was used in A and B. Significant differences between treatments are shown in the legend and specific differences between treatment for a given PQ concentration in the figure. Data from the three independent experiments in 9C was transformed by \hat{ }0.25 and \hat{ }0.75 to compare the effect of vehicle and CA77 on arrestin area or opsin counts, respectively followed by paired two tailed t test. * p<0.05 and **p<0.01.

FIG. 10. CA compounds prevent rd10 retinas degeneration. FIG. 10A. Ratio of the thickness of the outer nuclear layer (ONL) and inner nuclear layer (INL) in retinas of rd10 mice treated from P18 to P25 with daily i.p. injection of vehicle only or 40 mg/kg bw of CA77. n=8 (vehicle) and 9 (CA treated), from 3 independent experiments. FIG. 10B. Rod (transducin) and cone (opsin) markers in temporal central retina of rd10 treated as in A. Nuclei are highlighted with DAPI. FIG. 10C. Quantification of outer segment (OS) length measured in the whole retina with the markers used in B. n=4 areas per animal, 4 mice per condition. FIG. 10D. mRNA levels of rho in the same animals. n=10. FIG. 10E. Representative image of the immunostaining for GFAP in the same retinas. n=4. FIG. 10F. Immunoblot for the indicated proteins in retinas of mice treated as in A. 4 different mice are shown. Right: Densitometric quantification of L2A in n=4 different mice. Values are expressed as arbitrary units. FIG. 10G. Electroretinogram parameters at P33 of rd10 mice administered vehicle or CA77 as in A. Amplitude of the indicated waves is plotted. Individual values and mean+s.e.m. are shown. Two-way Anova followed by Bonferroni's multiple comparisons post-hoc test was used in A, and unpaired two-tailed t test in all others. * p<0.05 and **p<0.01.

FIG. 11. NCoR1 expression is reduced in experimental mouse models and patients with retinitis pigmentosa. FIG. 11A. Protein levels of NCoR1 in retinas from wild type (WT) and rd10 mice at the indicated postnatal (P) days. Data from 22. Values are expressed as Z score. n=4 mice per genotype and time. FIG. 11B. Immunoblot for NCoR1 in retinas of WT (2 mice) and rd10 (3 mice). Ponceau Staining is shown as loading control. FIG. 11C. Immunostaining for NCoR1 in whole mount retinas from wild type (WT) and rd10 mice at p25. Nuclei are highlighted with DAPI. Merged Dapi and NCoR1 images (top) or only NCoR1 image (bottom). Bottom: Higher magnification images of the boxed regions to show differences in NCoR1 levels in the inner nuclear layer (INL) (top) and in the ganglion cell layer (GCL) (bottom). FIG. 11D. NCoR1 mRNA levels in WT and rd10 mice treated from P18 to P25 with daily i.p. injection of vehicle only or 40 mg/kg bw of CA77. Values are expressed as folds WT vehicle. n=4 (WT) and 7 (rd10) mice. FIG. 11E. Heat map of the expression of the indicated genes of the CMA transcriptional network in retinal organoids from healthy (Control) and retinitis pigmentosa patients (RP) bearing the PDE6B mutation at the indicated days of organoid differentiation (FIG. 11D). For patient samples, D90-D180 display features of mid-state disease and D230 of late state disease. CMA index is shown at the bottom. Data from Gao, M. L., et al., Front Cell Dev Biol. (2020) 8: 128. FIG. 11F, 11G. CMA activation index (FIG. 11F) and ratio of NCoR1 to RARα mRNA levels (FIG. 11G) in the same samples as in e. RNA was isolated from 3-5 organoids from two independent differentiations. FIG. 11H. mRNA levels of NCoR1 (left), RARα (middle) and NCoR1 to RARα ratio in retinal organoids from healthy (Control) and retinitis pigmentosa patients (RP) bearing mutations in RP2 at D180. Data from Lane, A., et al., Stem Cell Reports (2020) 15: 67-79, n=3 individuals per diagnosis. Two-way Anova was used in 11A, One-way Anova with Tukey's multiple comparison post hoc test in 11D and paired t test in 11H. *p<0.05 and ** p<0.01.

### DETAILED DESCRIPTION

Prior to setting forth the invention in detail, it may be helpful to provide definitions of certain terms to be used in this disclosure. Compounds are described using standard nomenclature. Unless defined otherwise, all technical and scientific terms used herein have the same meaning as is commonly understood by one of skill in the art to which this invention belongs. Unless clearly contraindicated by the context each compound name includes the free acid or free base form of the compound as well as all pharmaceutically acceptable salts of the compound.

The terms “a” and “an” do not denote a limitation of quantity, but rather denote the presence of at least one of the referenced items. The term “or” means “and/or”. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

“Pharmaceutical compositions” are compositions comprising at least one active agent, such as a compound or salt of a CMA Activator, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs.

Stabilization of the interaction of a Retinoic Acid Receptor-alpha (RARα) and a corepressor, such as Nuclear Receptor Corepressor 1 (NCoR1), can be determined by any test suitable for determining increased stability of the RARα/compressor interaction. For example increased coimmunoprecipitation of RARα and the corepressor in the presence of a CMA activator over the coimmunoprecipitation of the RARα and the corepressor in absence of the CMA activator can indicate that the interaction of RARα and the corepressor is stabilized. Increased expression of genes associated with CMA Activation also indicates stabilization of the RARα/corepressor interaction.

“Upregulating CMA gene expression” means that expression of one or more gene associated with CMA is increased. For example, the expression of at least one effector or activator gene associated with CMA is increased. CMA gene expression can be upregulated in a subject administered a CMA activator relative to CMA gene expression prior to administration of the CMA activator. Upregulating CMA gene expression can mean that as least one effector gene selected from LAMP2A, HSC70, HSP90AA1, HSP90AB1, HSP40, EEF1A1, PHLPP1, and RAC1 is increased in a subject administered a CMA activator relative to the expression of the effector gene in the patient prior to administration of the CMA activator or the expression level of at least one activator gene selected from NFATC1, NCOR1, NFE2L2, NFR-2, RARα, and Rab11 is increased in a subject relative to the expression of the activator gene in the patient prior to administration of the CMA activator.

Inventors have identified a unique mechanism for selective activation of CMA. Inventors have found CMA Activators (CA) stabilize the interaction between retinoic acid receptor alpha—a known endogenous inhibitor of CMA—and its co-repressor NCoR1, resulting in changes of a discrete subset of the RARα transcriptional program that leads to selective CMA activation. CA molecules activate CMA in vivo and ameliorate retinal degeneration in a retinitis pigmentosa mouse model. This disclosure includes methods for preventing or treating retinal degeneration. Our findings reveal a mechanism for pharmacological targeting of CMA activation and provide a method for treating and or preventing retinal degeneration and other age-related degenerative processes.

Without wishing to be bound to any particular mechanism, comparative structural analysis and molecular dynamics suggested that CMA selectivity may be related with the preference of these novel small molecules to bind and stabilize and open H12 conformation of the RARα ligand binding domain, thereby favoring recruitment of corepressors. The small molecules are predicted to use a non-canonical binding mode compared to other RARα antagonists and agonists that commonly use a carboxyl group to form electrostatic interactions with the RARα ligand binding domain,

The new CMA activators demonstrate good biodistribution and pharmacokinetic properties favorable for peripheral and central nervous system targeting. These compounds stabilize the interaction of RARα with its corepressor NCoR1. This novel mechanism of action of the CMA activators leads to the selective regulation of only a discrete subset of the RARα transcriptional program, thus conferring them selectivity for CMA. We provide evidence that the compounds efficiently activate CMA in vivo without noticeable toxicity. We also demonstrate that in vivo administration of the novel CMA activators, either systemically or locally, e.g. intravitreal injection, efficiently reduces retinal degeneration and preserves visual function in an experimental mouse model of retinal degeneration of clinical relevance for retinitis pigmentosa, an incurable condition that results in blindness. This work provides proof of concept for pharmacologically targeting the transcriptional mechanism of CMA regulation in a retinal degenerative setting.

Currently, diagnosis of neurodegenerative disorder such as Alzheimer's disease (AD) relies on identifying mental decline, at which point significant brain damage has been done. Similarly, Parkinson's disease (PD) is identified by symptoms such as shaking or tremors, slowness of movement (bradykinesia), stiffness or rigidity of the arms and legs, and/or balance issues (postural instability). PD is a progressive disease in which the symptoms worsen over time. The methods described herein provide for preventing or slowing advancement of an age-related neurodegenerative disease in a subject in need thereof when the subject is asymptomatic or is in an early symptomatic stage of the age-related neurodegenerative disease. Early intervention may help to prevent the progression of symptoms and delay progression to late-stage age-related neurodegenerative disorder.

In an aspect, a method of preventing or slowing advancement of an age-related neurodegenerative disorder in a subject in need thereof comprises identifying an early symptom or biomarker of the neurodegenerative disorder in the subject, and administering a therapeutically effective amount of a CMA activator to the subject. In an aspect, the subject is asymptomatic or is in an early symptomatic stage of the age-related neurodegenerative disorder.

Administering the CMA activator can reduce the progression of beta-amyloid and/or tau pathology in the subject, and/or reduce pre-existing beta-amyloid and/or tau pathology in the subject. Prior to the experiments described herein, it was not expected that CMA modulation would affect beta-amyloid and/or tau pathology. The method optionally further comprises determining the progression of beta-amyloid and/or tau pathology by positron emission tomography (PET) and/or magnetic resonance (MR) imaging. ¹¹C-labeled Pittsburgh Compound-B ([¹¹C]PiB), also known as 2-(4-N-[¹¹C]methylaminophenyl)-6-hydroxybenzothiazole, [¹⁸F]Florbetapir ([¹⁸F]FBP), which is also known as ¹⁸F-AV-45 or 4-{(E)-2-[6-(2-{2-[2-(18F)Fluoroethoxy]ethoxy}ethoxy)-3-pyridinyl]vinyl}-N-methylaniline, [¹⁸F]Florbetaben ([¹⁸F]FBB), and [¹⁸F]Flutemetamol ([¹⁸F]FMT) are radiotracers for beta-amyloid {ET imaging. The PET ligand [¹⁸F]AV-1451 binds tau-positive inclusions. The levels of tau protein (total tau or phosphorylated tau) or beta-amyloid (e.g., Aβ42) in the plasma or cerebrospinal fluid (CSF) of the subject can also be used to determine the progression of beta-amyloid and/or tau pathology.

### Pharmaceutical Preparations

Compounds disclosed herein can be administered as the neat chemical, but are preferably administered as a pharmaceutical composition. Accordingly, the disclosure provides pharmaceutical compositions comprising a compound or pharmaceutically acceptable salt of a CMA activator, together with at least one pharmaceutically acceptable carrier. In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of a compound of a CMA Activator and optionally from about 0.1 mg to about 2000 mg, from about 10 mg to about 1000 mg, from about 100 mg to about 800 mg, or from about 200 mg to about 600 mg of an additional active agent in a unit dosage form.

Compounds disclosed herein may be administered orally, topically, parenterally, by inhalation or spray, sublingually, transdermally, via buccal administration, rectally, as an ophthalmic solution, through intravitreal injection or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution for topical or intravitreal injection. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert, or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present disclosure.

The pharmaceutical compositions/combinations can be formulated for oral administration. These compositions contain between 0.1 and 99 weight % (wt. %) of a CMA Activator and usually at least about 5 wt. % of a CMA Activator. Some embodiments contain from about 25 wt. % to about 50 wt. % or from about 5 wt. % to about 75 wt. % of the compound of Formula.

### Methods of Treatment

The disclosure also provides methods of selectively activating chaperone-mediated autophagy (CMA) in a subject in need thereof comprising administering to the subject a CMA Activator in an amount effective to activate CMA in the subject.

The subject can have, for example, a neurodegenerative disease, such as tauopathies, (Frontotemporal Dementia, Alzheimer's disease), Parkinson's Disease, Huntington's Disease, prion diseases, amyotrophic lateral sclerosis, retinal degeneration (dry or wet macular degeneration, retinitis pigmentosa, diabetic retinopathy, glaucoma, Leber congenital amaurosis), diabetes, acute liver failure, non-alcoholic steatobepatitis (NASH), hepatosteatosis, alcoholic fatty liver, renal failure and chronic kidney disease, emphysema, sporadic inclusion body myositis, spinal cord injury, traumatic brain injury, fibrosis (liver, kidney, or lung), a lysosomal storage disorder, a cardiovascular disease, and immunosenescence. Lysosomal storage disorders include, but are not limited to, cystinosis, galactosialidosis, and mucolipidosis. The subject may also have a disease or condition in which CMA is upregulated such as cancer or Lupus. The subject can have reduced CMA compared to a normal subject prior to administering the compound. Preferably, the compound does not affect macroautophagy or other autophagic pathways. In macroautophagy, proteins and organelles are sequestered in double-membrane vesicles and delivered to lysosomes for degradation. In CMA, protein substrates are selectively identified and targeted to the lysosome via interactions with a cytosolic chaperone and cross the lysosomal membrane through a translocation complex.

The disclosure also provides a method of protecting cells from oxidative stress, hypoxia, proteotoxicity, genotoxic insults or damage and/or lipotoxicity in a subject in need thereof comprising administering to the subject any of the compounds disclosed herein, or a combination of a CMA Activator, in an amount effective to protect cells from oxidative stress, hypoxia proteotoxicity, genotoxic insults or damage, and/or lipotoxicity. The subject can have, for example, one or more of the chronic conditions that have been associated with increased oxidative stress and oxidation and a background of propensity to proteotoxicity. The cells being protected can comprise, for example, cardiac cells, kidney and liver cells, neurons and glia, myocytes, fibroblasts and/or immune cells. The compound can, for example, selectively activate chaperone-mediated autophagy (CMA). In one embodiment, the compound does not affect macroautophagy.

In a specific aspect, the subject is suffering from mild cognitive impairment. As used herein, mild cognitive impairment is the stage between the expected cognitive decline due to aging and the more serious decline of dementia. Forgetfulness, losing train of thought or difficulty following conversations, difficulty making decisions, getting lost in familiar environments and poor judgment can be signs of mild cognitive impairment. Mild cognitive impairment can progress to Alzheimer's disease or other forms of dementia.

Exemplary age-related neurodegenerative diseases include Alzheimer's disease (AD), Lewy body dementia, Parkinson's disease (PD), Huntington's disease, Amyotrophic lateral sclerosis (ALS), Frontotemporal dementia (FTD), Spinocerebellar ataxias (SCAs), Progressive subcortical gliosis, and the like.

When the age-related neurodegenerative disease is AD, the subject for the methods described herein subject may not suffer from dementia. Exemplary early symptoms of AD include memory loss and/or confusion, difficulty concentrating, difficulty completing daily tasks, time and/or place confusion, difficulty with visual images and/or spatial relationships, difficulty conversing, misplacing objects, poor judgment, withdrawal from activities, changes in mood and personality. Exemplary biomarkers for AD are tau protein (total tau or phosphorylated tau) or beta-amyloid (e.g., Aβ42) in the plasma or cerebrospinal fluid (CSF) of the subject.

In Lewy body dementia, protein deposits called Lewy bodies develop in nerve cells in the regions of the brain involved in cognition, memory, and movement. Early symptoms of Lewy body dementia include loss of small, acting out while dreaming, visual hallucinations, confusion, difficulty maintaining attention, memory loss, changes in handwriting, muscle rigidity, falling, and drowsiness. Currently there are no verified biomarkers for Lewy body dementia.

PD is a progressive nervous system disorder that affects movement. Exemplary early symptoms of PD include slight tremors in the fingers, thumbs, hand or chin; small handwriting (also called micrographia); loss of smell; difficulty sleeping including sudden movements in sleep; difficulty moving or walking; constipation; a soft or low voice; facial masking; dizziness or fainting; and/or stooping, leaning or slouching while standing. Currently there are no verified clinical biomarkers for PD.

Huntington's disease is a genetic disorder that causes progressive degeneration of nerve cells in the brain. Early symptoms of Huntington's disease include difficulty concentrating, memory lapses, depression, clumsiness, small involuntary movements and mood swings. Mutant Huntington protein (mHtt) is a biomaker for Huntington's disease. Subjects who carry the Huntington mutation can be treated by the methods described herein.

ALS is a rare, progressive disease involving the nerve cells responsible for controlling voluntary movements. Early symptoms of ALS include muscle twitches in the arm, leg, shoulder or tongue; muscle cramps; stiff muscles; muscle weakness of the arm, leg, neck or diaphragm; slurred and nasal speech; and difficultly chewing or swallowing. Currently there are no validated biomarkers for ALS.

FTD, sometimes called Pick′ disease, is a group of neurological disorders in which nerve cells in the front and temporal lobes of the brain are lost. Early symptoms of FTD include changes to personality and behavior and/or difficulties with language. Clinically, differentiating between FTD and AD is challenging.

Spinocerebellar ataxias (SCAs) are progressive disorders in which the cerebellum slowly degenerates, often accompanied by degenerative changes in the brainstem and other parts of the central nervous system. Early symptoms of SCAs are problems with coordination and balance, speech and swallowing difficulties, muscle stiffness, weakness of the muscles that control eye movement, and cognitive impairment. SCA1, SCA2, SCA3, SCA6, SCA7 and SCA17 share the same pathogenic mechanism of CAG trinucleotide repeat expansions encoding elongated polyglutamine tracts. There are no serum biomarker for SCAs.

The disclosure also provides a method of treating a subject at risk for a neurodegenerative disorder. A subject at risk for a neurogenerative disorder has significantly greater probability of developing the neurodegenerative disorder than the prevalence of the disorder indicates the probability of developing the disorder would be. For example if 1 in 1000 people develop the disorder, then the probability of developing the disorder is 0.1%. A subject having a risk factor for developing the disorder would have greater than a 0.1% probability of developing the disorder. Risk factors can include genetic risk factor such as having a genetic mutation known to be associated with developing the neurodegenerative disorders, environmental risk factors, and lifestyle risk factors.

In an embodiment the subject is a mammal. In certain embodiments the subject is a human, for example a human patient undergoing medical treatment. The subject may also be a companion a non-human mammal, such as a companion animal, e.g. cats and dogs, or a livestock animal.

For diagnostic or research applications, a wide variety of mammals will be suitable subjects including rodents (e.g. mice, rats, hamsters), rabbits, primates, and swine such as inbred pigs and the like. Additionally, for in vitro applications, such as in vitro diagnostic and research applications, body fluids (e.g., blood, plasma, serum, cellular interstitial fluid, cerebrospinal fluid, saliva, feces and urine) and cell and tissue samples of the above subjects will be suitable for use.

An effective amount of a pharmaceutical composition may be an amount sufficient to inhibit the progression of a disease or disorder, cause a regression of a disease or disorder, reduce symptoms of a disease or disorder, or significantly alter a level of a marker of a disease or disorder.

An effective amount of a compound or pharmaceutical composition described herein will also provide a sufficient concentration of a CMA Activator when administered to a subject. A sufficient concentration is a concentration of the CMA Activator in the patient's body necessary to prevent or combat a CMA mediated disease or disorder or other disease ore disorder for which a CMA Activator is effective. Such an amount may be ascertained experimentally, for example by assaying blood concentration of the compound, or theoretically, by calculating bioavailability.

Methods of treatment include providing certain dosage amounts of a CMA Activator to a subject or patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a CMA Activator are provided daily to a patient. Frequency of dosage may also vary depending on the compound used and the particular disease treated. However, for treatment of most diseases and disorders, a dosage regimen of 4 times daily or less can be used and in certain embodiments a dosage regimen of 1 or 2 times daily is used.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

In an embodiment, the invention provides a method of treating a lysosomal storage disorder in a patient identified as in need of such treatment, the method comprising providing to the patient an effective amount of a CMA Activator. CMA Activators may be administered alone as the only active agent, or in combination with one or more other active agent.

### EXAMPLES

### General Methods

### Animal, Cells and Reagents

Animals: C57BL/6J mice wild-type (WT) and homozygous for the Pde6 mutation (rd10 mouse model of retinal degeneration) were obtained from The Jackson Laboratory. C57BL/6 KFERQ-Dendra mice were generated by back-crossing FVB KFERQ-Dendra mice with wild-type C57BL/6 mice for 8 generations. Both male and female animals were used in this study in equal distribution groups. However, results from both sexes were pooled, because of absence of significant differences in any of the parameters analyzed after statistical analysis with a mixed model of two-way ANOVA sex and treatment as independent variables and the corresponding measure as dependent variable. All animals were housed in a barrier-controlled facility (19-23° C. 30-60% relative humidity; 12-h light/dark cycle) with ad libitum access to standard chow pellets and water. For animal administration of CA compounds, a formula containing 30% PEG 400, 65% glucose solution (5%), 5% Tween 80 was used to dissolve them. CA compounds were prepared freshly by adding 5% DMSO and sonicating for 1 h. Treatment of KFERQ-Dendra mice with the CA compounds was done by i.p. daily injection of 30 mg/kg bw or vehicle for three consecutive days and tissues collected 6 h after the last injection. In the case of rd10 mice, animals were daily injected from P18 to P25 with CA77 (40 mg/kg bw). At P26, mice were sacrificed, and eyes fixed overnight with 4% PFA in PBS at 4° C. for immunofluorescence or retinas dissected and immediately frozen for biochemistry. Distribution of animals in the vehicle or treatment group was done randomly. All animal studies and procedures complied with ethical regulations, were performed in accordance with the European Union guidelines and the ARVO Statement for the Use of Animals in Ophthalmic and Vision Research and were approved by the Institutional Animal Care and Use Committee at the Albert Einstein College of Medicine, the CSIC Bioetica Comite and approved by the Comunidad de Madrid, PROEX232/17.

Cells: NIH3T3 mouse fibroblasts and the N2a neuroblastoma cell line were obtained from the American Type Culture Collection and were validated by genomic PCR. Primary human fibroblasts (GM01651) were from Coriell Repository. All the cells lines were tested for mycoplasma contamination using DNA staining protocol with Hoechst 33258 dye. Knock-down of NCoR1 was done using lentiviral mediated shRNA (SHCLNG-NM_011308) from the Sigma Mission library following standard procedures. Efficiency of knock-down was tested by immunoblot. Cell viability was measured using the CellTiter-Blue kit (Promega) 24 h after the addition of the different stressors according to manufacturer's instructions.

Antibodies: Primary antibodies were from the following sources: (dilution for use and clone indicated in brackets): rabbit anti LC3B (1/1000, MBL pm036), mouse anti β-actin (1/10000 Sigma, A4700), mouse anti MAP2 (1/1000, Sigma-Aldrich, M1406), rabbit anti GFAP (1/1000, DAKO, Z0334), rabbit anti RARα (1/1000, Cell Signaling, 2554), mouse anti visual arrestin (1/200, Santacruz Biotechnologies, C-3, Sc-166383) and rabbit anti Opsin R/G (1/1000, Millipore, AB5405), rabbit anti trasducin (1/200, Santacruz Biotechnologies, sc-389), rabbit anti NCoR1 (1/100, Cell Signaling, 5948), rabbit anti L2A (1/2000, Invitrogen, 51-2200). All the secondary antibodies were purchased from Thermo Scientific. All antibodies used in this study were from commercial sources and were validated following the multiple dilution method and where available through the use of cell lines or tissues from animals knock-out for the antigen.

### Human Retinal Tissue

Data from human tissue from healthy individuals and retinitis pigmentosa patients was obtained from previous published studies and no recruitment or collection of human tissue was performed for the completion of this work. Sources of the human tissues for the generation of the retinal organoids are detailed in the original studies. (Gao, M. L, et. al., Front. Cell Dev. Biol., (2020) 8: 128, Lane, A., et al., Stem Cell Reports, (2020) 15: 67-79) Briefly, in Gao patient mononuclear cells were collected and subjected to a plasmid-based reprogramming system to generate human iPSCs that were subsequently subjected to protocols for trilineage differentiation. In Lane, consent skin biopsies were obtained to isolate dermal fibroblasts and iPSCs were generated from two unrelated RP patients and 2 controls. In both studies, collection of human samples was approved by the research ethics committees in their respective countries.

### Autophagic Measurements

Intracellular proteolysis of long half-life proteins: Confluent cells were labelled with [³H]leucine (2 μCi/ml) (NEN-PerkinElmer Life Sciences) for 48 h and then extensively washed and maintained in medium with an excess of unlabeled leucine. Proteolysis was calculated from aliquots of the medium taken at different times and precipitated in trichloroacetic acid, as the amount of acid-precipitable radioactivity transformed to acid-soluble radioactivity at each time.

Macroautophagy activity: Cells were transduced with a lentiviral vector expressing mCherry-GPF-LC3³², fixed and flux determined as the conversion of dual fluorescence puncta (autophagosomes) into only red fluorescent puncta (autolysosomes). Flux was also measured by immunoblot for LC3 in cells incubated for 6 h with 20 mM NH₄Cl/100 μM leupeptin, by discounting the intensity of LC3 in non-treated cells from that in cells treated with the inhibitors.

CMA activity: Cells were transduced with lentivirus carrying the KFERQ-PS-Dendra reporter as before. Cells were photoactivated by exposure to a 3.5 mA (current constant) light emitting diode (LED: Norlux, 405 nm) for 3 min and then plated in glass-bottom 96 well plates. At the desired times, cells were fixed with 4% PFA and imaged using high-content microscopy (Operetta system, Perkin Elmer). Images were quantified using the manufacturer's software in a minimum of 1,200 cells in 9 independent fields per well. Although cell lines used in this study (NIH 3T3 and N2a) stably express the fluorescent reporter and the percentage of transduced cells is usually >85%, we set the program to identify number of cells by nuclei but to discount nuclei that did not have associated cytosolic green fluorescence. This was particularly important in the case of primary human cells where efficiency of transduction was approximately 65%. To avoid con-funding effects for drug-induced changes in cell volume or adherence to the plate that could bring a fraction of the cells per field outside of focus, we set a threshold to count only cells that have at least 1/10 of the average number of cells detected in the untreated wells for each cell type (3-4 puncta/cell in the case of NIH3T3 and N2a cells and 5 puncta/cell in primary human fibroblasts). To measure CMA in the tissues from KFERQ-Dendra mice, the organs of interest were fixed for 12 h at 4° C. in picric acid fixation buffer (2% formaldehyde, 0.2% picric acid in PBS, pH7.0) and then washed with 70% ethanol, followed by two washes in PBS. Tissues were immersed in 30% sucrose and then embedded in OCT for sectioning in a cryostat (Leica CM3050 S). After airdrying for 30 min, sections were stored at −20° C. until use. Slices were mounted in DAPI-Fluoromount-G to highlight the cell nucleus and allow quantification of puncta per cell. Images were acquired in x-y-z planes with an Axiovert 200 fluorescence microscope (Carl Zeiss Ltd) with a ×63 objective and 1.4 numerical aperture, mounted with an ApoTome.2 slider or with a Confocal microscope (TCS SP5; Leica) using an HCX Plan Apo CS×63.0 1.40 NA oil objective and the Leica Application Suite X (LAS X).

### Chemical Synthesis of CMA Activators

All chemical reagents and solvents were obtained from commercial sources (Aldrich, Acros, Fisher) and used without further purification unless otherwise noted. Chromatography was performed on a Teledyne ISCO CombiFlash Rf 200i using disposable silica cartridges (4, 12, and 24 g). Analytical thin layer chromatography (TLC) was performed on aluminum-backed Silicycle silica gel plates (250 μm film thickness, indicator F254). Compounds were visualized using a dual wavelength (254 and 365 nm) UV lamp, and/or staining with CAM (cerium ammonium molybdate) or KMnO₄ stains. NMR spectra were recorded on Bruker DRX 300 and DRX 600 spectrometers. ¹H and ¹³C chemical shifts (δ) are reported relative to tetramethyl silane (TMS, 0.00/0.00 ppm) as internal standard or to residual solvent (CDCl₃: 7.26/77.16 ppm; dmso-d6: 2.50/39.52 ppm). Mass spectra (ESI-MS) were recorded on a Shimadzu LCMS 2010EV (direct injection unless otherwise noted). High resolution mass spectra (HRMS) were recorded on an Orbitrap Velos high resolution mass spectrometer at the Proteomics Facility of Albert Einstein College of Medicine.

The synthesis of AR7, CA39, and CA77 was disclosed previously.

### Expression and Purification of RARα LBD.

The histidine-tagged ligand binding domain (LBD) of human RARα (residues 176-421) was expressed in Escherichia colic BL21(DE3). Cells were grown at 37° C. in LB medium supplemented with 50 μg/mL kanamycin until OD600 reached about 0.8. Expression of T7 polymerase was induced by addition of isopropyl-b-d-thiogalactoside (IPTG) to a final concentration of 0.8 mM and cells were incubated at 20° C. overnight. Cell cultures were harvested by centrifugation at 8,000×g for 15 mins. The cell pellet from 1 liter of RARα LBD was resuspended in 50 mL lysis buffer (20 mM Tris-HCl pH 8, 500 mM NaCl, 25 mM imidazole) supplemented with ONE COMPLETE, an EDTA-free protease inhibitor tablet (Roche Applied Science). The suspension was lysed using a high-pressure homogenizer and centrifuged at 35,000×g at 4° C. for 45 minutes. The supernatant was filtered and loaded onto a prepared 5 ml Ni²⁺-affinity column (HIS PUR Ni-NTA resin, THERMO SCIENTIFIC), preequilibrated with lysis buffer. The column was washed 3× with 15 mL lysis buffer. Bound proteins were eluted with lysis buffer containing 200 mM imidazole. Eluted protein was concentrated, and buffer exchanged in FPLC buffer (10 mM HEPES, 150 mM NaCl, pH8) using an AMICON Ultra-15 10K centrifugal filter unit (Millipore Sigma). The protein was future purified using a Superdex 200 Increase 10/300 GL gel filtration column (Fisher Scientific) preequilibrated with FPLC buffer. Purified RARα fractions were pooled, 5 mM DTT was added, and protein was stored at 4° C.

### Fluorescence Polarization Binding Assays

The fluorescein-tagged peptide of NCoR1, FITC-Ahx-RLITLADHICQIITQDFAR (FITC-NCoR1) was provided by Genscript at purity>95%. Fluorescence polarization assays (FPA) were performed using established procedures. Direct binding isotherms were generated by incubating FITC-NCoR1 (5 nM) with or without small molecules (10 μM) with serial dilutions of RARα LBD starting from 10 μM and diluted two fold at each step. The buffer solution for assays was 20 mM Tris-HCl, pH 7.5, 150 mM NaCl, 1 mM EDTA, 5 mM DTT and 10% (v/v) glycerol. Fluorescence polarization was measured at 30 min on a F200 PRO microplate reader (TECAN) with the excitation wavelength set at 470 nm and emission measured at 530 nm. EC50 values were calculated by nonlinear regression analysis of competitive binding curves using Graphpad Prism software.

### Molecular Docking and Structural Analysis

AR7, CA39, and CA77 structures were drawn in ChemDraw and converted to three-dimensional all-atom structures from sdf format using LigPrep (Schrödinger, LLC). For each ligand a maximum of 4 stereoisomers were generated, ionization states and tautomers were generated for pH 7 and pH 2, geometries optimized, and energy minimized before docking. The structure of the RARα-RXR hetero-dimer in complex with the small molecule antagonist BM614 (PDB ID: 1DKF), was used for docking. The RARα-RXR structure was prepared using MAESTRO protein preparations module (Schrödinger, LLC). The structure of the antagonist was removed from the RARα site, water molecules at a distance of more than 5 Å from heteroatoms were removed, all missing protons were generated, hydrogens were optimized for best hydrogen bonding network bonds and formal charges were assigned and structure was gently minimized by restrained energy minimization. The ligand-binding pocket was defined within 5 Å of the BMS614 pose, and receptor grid size and center was generated based on the position and the size of the BMS614. To account for receptor flexibility in docking, scaling of van der Waals' radii of non-polar atoms with the absolute value of the partial atomic charge less than or equal to 0.25 for protein atoms was set to 1 and for ligand non-polar atoms with partial atomic charges less than or equal to 0.15 was set to 0.8. Docking was performed in ligand flexible mode using Glide (Schrödinger, LLC) using the extra precision (XP) mode. All three molecules were docked into the BMS614 binding site. Structures were analyzed using MAESTRO and PyMOL (Schrödinger, LLC.)

### In Vitro and in Silico ADME

In vitro solubility in the following conditions A) HBSS/HEPES 10 mM/BSA 0.1% pH 6 B) HBSS/HEPES 10 mM/BSA 0.1% pH 7.4 C) TRIS/BSA 0.1% PBS (pH 7.4 and at indicated doses were measured by Nephelometry using the NEPHELOstar Galaxy apparatus (BMG Lab Technologies). In vitro stability in human, mouse and rat liver microsomes and permeability using a bidirectional permeability assay with CaCo-2 cells (pH 6.5/7.4) assays were performed with standard methodology and analyzed by LC-MS/MS. at SIMM-SERVIER joint Biopharmacy Laboratory. In silico ADME properties calculated by QikProp (Schrodinger, LLC) analysis and ADME predictions for CA39 (left) and CA77 (right).

### Pharmacokinetic Analysis

ICR (CD-1) male mice were fasted at least three hours and water was available ad libitum before the study. Animals were housed in a controlled environment, target conditions: temperature 18 to 29° C., relative humidity 30 to 70%. Temperature and relative humidity was monitored daily. An electronic time controlled lighting system was used to provide a 12 hr light/12 hr dark cycle. 3 mice for each indicated time point were administered 30 mg/Kg CA39 or CA77 by oral gavage or 1 mg/Kg CA39 or CA77 by intravenous injection using 30% PEG-400, 65% D5W (5% dextrose in water), 5% Tween-80 vehicle. Mice were sacrificed, and brain samples were harvested at 0 hr, 0.25 hr, 0.5 hr, 1 hr, 2 hr, 4 hr, 8 hr, 24 hr, and analyzed for CA39 or CA77 levels using LC-MS/MS. Pharmacokinetics parameters were calculated using Phoenix WinNonlin 6.3. Experiments performed at SIMM-SERVIER joint Biopharmacy Laboratory.

### Retina Processing and Staining

Ex-vivo retinal cultures: After removal of the eyes, all non-relevant tissue was removed from the neuroretina, which was then placed, photoreceptors upside down, in millicell support inserts (Millipore) and maintained in DMEM with 1 μM insulin (SIGMA, I2643-25 mg) for 24 h at 37° C. in a 5% CO₂ atmosphere. Where indicated, retinas were incubated with 10 □M CA77 for the indicated times. The retinas were then washed twice with phosphate-buffered saline (PBS), fixed overnight in 4% paraformaldehyde (w/v) in 0.1 M phosphate buffer (pH 7.4) and processed.

Staining of whole-mount retinas: Immunostainings were performed overnight at 4° ° C. using antibodies against visual arrestin (Santacruz Biotechnologies) and Opsin R/G (Millipore) after initial permeabilization with 2% Triton X-100 for 1 h at RT and subsequent incubation with blocking solution (10% normal goat serum, 0.25% Triton X-100 in PBS). The retinas were then washed and incubated for 1 h with Alexa 568 or 647 (Invitrogen), counterstained with DAPI, mounted in Fluoromount, and visualized by confocal microscopy in a SP5 confocal microscope (TCS SP5; Leica Microsystems).

Cryosections and immunofluorescence: Cryosections and immunofluorescence in retinal sections were performed as previously described 10. Primary antibodies used in this study were Transducin (Santacruz Biotechnologies) and Opsin R/G (Millipore). Sections were visualized by confocal microscopy (TCS SP5; Leica Microsystems).

ONL thickness and outer segment length quantification: For ONL thickness quantification, DAPI images were taken at 40× in a fluorescence microscope Multidimensional system Leica AF6000 LX coupled to DMI600B microscope and Hamamatsu CCD 9100-02 camera. At least two sections per animal were analyzed, preferably central sections. Eight images per retina equally distributed along the retina were acquired. Three measures were taken per image and ratio ONL/INL quantified with ImageJ tools. Leica LAS-X was used for image acquisition and ImageJ (v.2.1.0) was used for image processing. For OS length measures, fixed positions common to all pictures were considered and OS length was measured using ImageJ straight line tool.

### ERG Recordings

Mice were dark adapted overnight, and subsequent manipulations were performed in dim red light. Mice were anesthetized with intraperitoneal injections of ketamine (95 mg/kg) and xylazine (5 mg/kg) solution and maintained on a heating pad at 37° C. Pupils were dilated with a drop of 1% tropicamide (Colircusi Tropicamida; Alcon Cusi). To optimize electrical recording, a topical drop (2% Methocel; Hetlingen) was instilled on each eye immediately before situating the corneal electrode. Flash-induced ERG responses were recorded from the right eye in response to light stimuli produced with a Ganzfeld stimulator. Light intensity was measured with a photometer at the level of the eye (Mavo Monitor USB; Nürenberg). Four to 64 consecutive stimuli were averaged with an interval between light flashes in scotopic conditions of 10 s for dim flashes and of up to 60 s for the highest intensity. Under photopic conditions, the interval between light flashes was fixed at 1 s. ERG signals were amplified and band-filtered between 0.3 and 1000 Hz with an amplifier (CP511 AC amplifier; Grass Instruments). Electrical signals were digitized at 20 kHz with a power laboratory data acquisition board (AD Instruments). Bipolar recording was performed between an electrode fixed on a corneal lens (Burian-Allen electrode; Hansen Ophthalmic Development Laboratory) and a reference electrode located in the mouth, with a ground electrode located in the tail. Under dark adaptation, scotopic threshold responses (STR) were recorded to light flashes of −4 log cd·s·m⁻²; rod responses were recorded to light flashes of −2 log cd·s·m⁻² s and mixed responses were recorded in response to light flashes of 1.5 log cd·s·m⁻². Oscillatory potential (OP) was isolated using white flashes of 1.5 log cd·s·m⁻² in a recording frequency range of 100 to 10,000 Hz. Under light adaptation, cone-mediated responses to light flashes of 2 log cd·s·m⁻² on a rod-saturating background of 30 cd·m⁻² were recorded. Wave amplitudes of the STR, rod responses (b-rod), mixed responses (a-mixed and b-mixed) and OP were measured offline by an observer masked to the experimental condition of the animal.

mRNA Quantification

mRNA-qPCR: RNA was extracted from individual retinas. Total RNA from retinas was extracted using TRIzol Reagent (Invitrogen), and reverse transcription performed using the High-Capacity cDNA Reverse Transcription Kit (Applied Biosystems) according to the manufacturer's instructions. Quantitative real-time PCR was performed in a Light Cycler 480 Instrument (Roche) with Taqman Universal PCR Master Mix using Taqman assays (Life Tech-nologies). The following probes were used: Mm01184405_m1 (rhodopsin), F-5′-CTTAGCTTCTGGGATGC CCC-3′, R-5′-GCACTGCAGTCTTGAGCTGT-3′ (lamp2a) F-5′-AA GGACTCCTA TAGTGGG TGACGA-3′, R-5′-ATCTTCTCCATGTCGTCCCAGTTG-3′ (mouse β-actin).

Microarray: Total RNA of the treated cells was extracted using TRIzol (Invitrogen) and purified with RNeasy chromatography (Qiagen). Cy3-labeled RNA (0.6 μg) from each condition were hybridized to Agilent Mouse 8×60K. Data were processed using the oligo package and normalized using Robust Multiarray Average (RMA) method. Gene set was filtered to remove genes without Entrez or GO annotation (21912 genes out of 55682) and genes with an IQR>0.5. The full microarray Gomez-Sintes et al. raw data will be deposited in GEO upon acceptance of the manuscript. Pathway analysis was performed using the STRING database (https://string700db.org/).

### Co-Immunoprecipitation and Immunoblot

Co-immunoprecipitation: Cells were lysed in 25 mM Tris, pH 7.2, 150 mM NaCl, 5 mM MgCl₂, 0.5% NP-40, 1 mM DTT, 5% glycerol and protease inhibitors for 15 min on ice and then centrifuged for 15 min at 16,000 g. Supernatant were precleared with Protein A/G sepharose and then incubated with the primary antibody overnight at 4° C. with continuous rocking. Protein A/G sepharose was added to the tubes and after incubation in the same conditions for 1 h, samples were spun and supernatant (FT, flow through) and beads (IP, immunoprecipitate) were subjected to SDS-PAGE and immunoblot.

Immunoblot: Cells were lysed in RIPA and neuroretinas in a buffer containing 50 mM Tris-HCl (pH 6.8), 10% glycerol (v/v), 2% SDS (w/v), 10 mM DTT, and 0.005% bromophenol blue. Protein concentration was determined using the Lowry method with bovine serum albumin as standard. Fifty micrograms of protein (for cell lysates) or 15 micrograms of protein for neuroretinas were resolved on AnyKD SDS-PAGE gel (BioRad). The proteins were then transferred to PVDF membranes (Bio-Rad), which were blocked for 1 h in PBS-Tween 20 (0.05% (v/v)) containing 5% non-fat milk and then probed with primary and secondary antibodies. Antigen signals were detected using the appropriate horseradish peroxidase-labelled secondary antibodies (Pierce) and were visualized with the SuperSignal West Pico chemiluminescent substrate (Pierce). Densitometric analysis was performed with Quantity One software (Bio-Rad).

### Histopathology of Peripheral Organs and Blood Cell Count

Liver, lung and kidneys from CA-treated animals were dissected and fixed in 1% PFA overnight and paraffin embedded. Tissues were sectioned, stained with hematoxylin and eosin (H&E) and analyzed by an expert pathologist, blind to the treatment groups, to score for possible presence of toxicity in these organs. Scoring 1-6 was used assigning 6 to those observations clinically relevant. Individual scoring per parameter and per organ and average scoring per organ were performed. Blood cell count in the groups of mice administered vehicle or CA was analyzed in tail blood drawn monthly and at the moment of tissue dissection using an Oxford Science Forcyte Blood Analysis Unit.

### Calculation of CMA Activation Index

CMA index was calculated using data set from Ly (J. Proteome Research (2016) 15: 1350-1359), Gao (2020), and Lane (2020). Briefly, each element of the CMA network was attributed a weight. As LAMP-2A is the rate limiting component of CMA, it was given a weight of 2. Every other element received a weight of 1. Then, every element was attributed direction score that is +1 or −1 based on the known effect of a given element on CMA activity. The score was then calculated as the weighted/directed average of expression counts of every element of the CMA network.

### Statistical Analysis and Sample Size Determination

All numerical results are reported as mean+s.e.m. and represent data from a minimum of three independent experiments unless otherwise stated. Statistical significance of difference between groups was determined in instances of single comparisons by the two-tailed unpaired Student's t-test of the means. In instances of multiple means comparisons, we used one-way analysis of variance (ANOVA) followed by the Bonferroni post hoc test to determine statistical significance. In the studies of resistance to oxidative stress in contralateral retinas treated with vehicle or CA, data transformation was applied as indicated in the legend of the figure. Statistical analysis was performed in all of the assays, and significant differences are noted in the graphical representations. If assumptions of normality and homoscedasticity were not met, we applied non-parametric tests. In studies with data normalized over control we used one sample t test with hypothesized control mean of 1. For all tests the significance level was p<0.05 (2-tailed). The number of animals used per experiment was calculated through power analysis based on previous results. Animals were randomly attributed to each treatment group using the “SELECT BETWEEN RANGE” function in Microsoft Excel. No mouse was excluded from the analysis unless there was technical reason, or the mouse was determined to be in very poor health by the veterinarian. For the studies involving cells in culture treatment groups were attributed randomly between wells and plates to account for well or tube positioning effects. We determine number of experimental repetitions to account for technical variability and changes in culture conditions based on our previous studies using those systems. Every experiment was performed in at least 3 independent replicates. Experiments in cells in culture were performed in different days to confirm reproducibility of the procedures. All independent replications were successful. Outliers were determined by the ROUT method (Q=1%). Investigators were blinded to the treatment during data collection and analysis and unblinding was done when the analysis was completed for plotting. Basic data handling was done in Microsoft Excel 365 (v.2101). Data analysis was performed with Prism software (v9-Graph Pad Software Inc). Image analysis and quantification was performed using ImageJ (v.2.1.0).

### Example 1. Novel Small Molecules Targeting RARα Promote Selective CMA Activation

AR7, reported previously, binds to the RARα ligand binding domain (LBD) and selectively activates CMA in vitro. (Anguiano, J., et al., Nat. Chem. Biol. (2013) 9: 374-382.) CA39 and CA77, also previously reported, are more potent activators of CMA (>40% CMA of AR7), without noticeable toxicity and capable to upregulate both basal and inducible CMA.

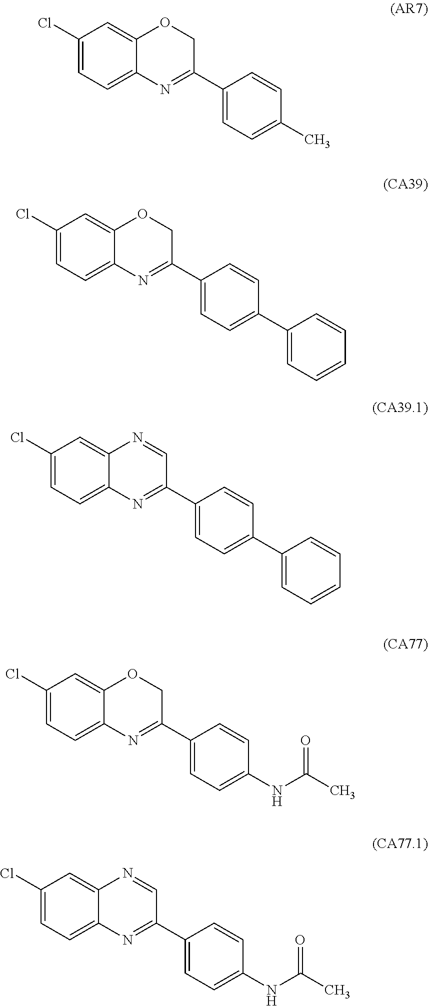 [SMILES: Cc1ccc(C2=Nc3ccc(Cl)cc3OC2)cc1 ; Clc1ccc2c(c1)OCC(c1ccc(-c3ccccc3)cc1)=N2 ; Clc1ccc2nc(-c3ccc(-c4ccccc4)cc3)cnc2c1 ; CC(=O)Nc1ccc(C2=Nc3ccc(Cl)cc3OC2)cc1 ; CC(=O)Nc1ccc(-c2cnc3cc(Cl)ccc3n2)cc1]

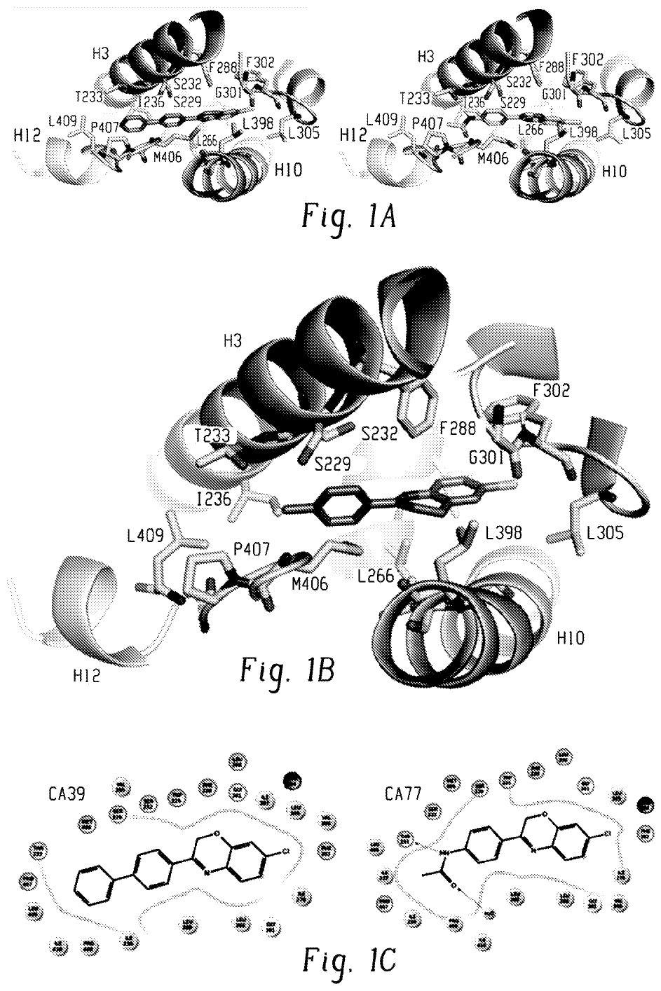

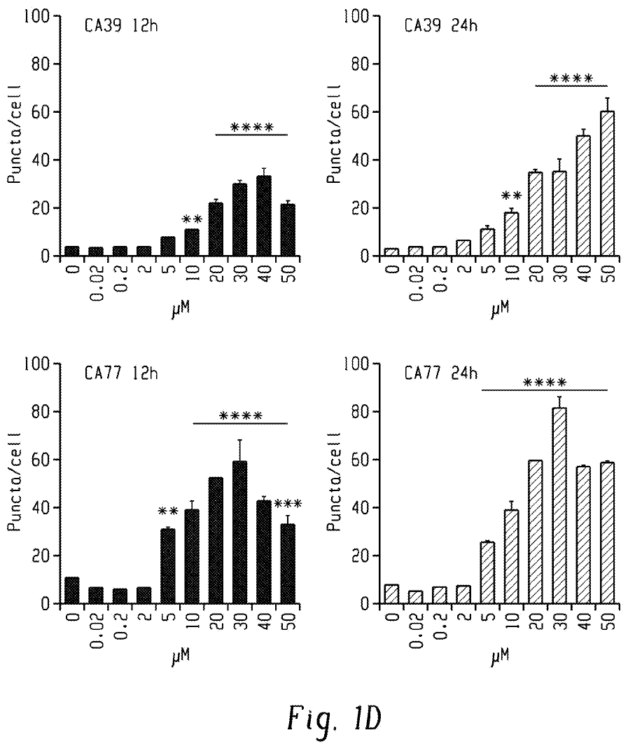

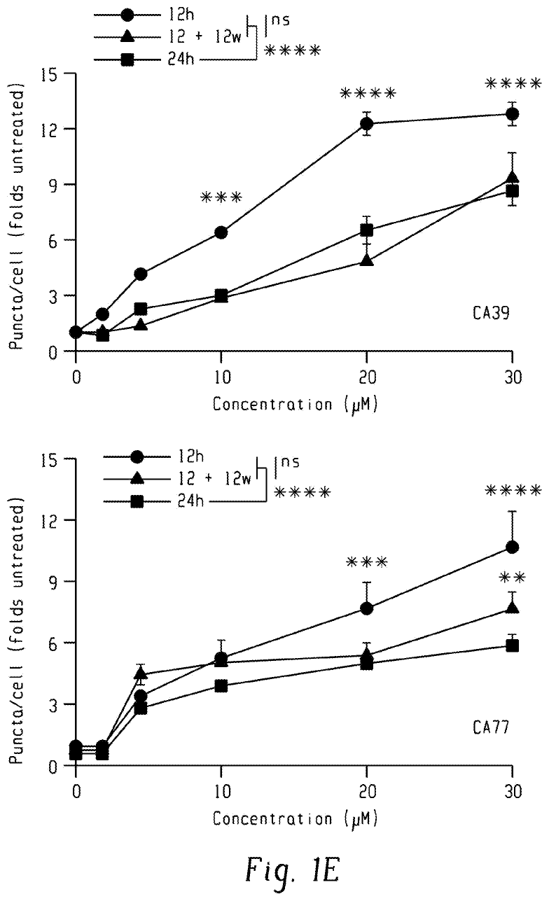

Molecular docking studies, performed as described herein, of CA39 and CA77 consistently favored binding in the RARα-binding pocket formed by the junction of helices h3, h10 and h12 stabilizing the h12 in the open conformation that regulates recruitment of co-repressors and co-activators to RARα, similarly to AR7 (FIGS. 1A, 1B). CA39 and CA77 were designed based on the benzoxazine scaffold to increase hydrophobic interactions or polar interactions with the RARα pocket residues respectively. Indeed, both compounds form extensive hydrophobic contacts and CA77 hydrogen bonding between its amide group with Thr233 and a water molecule near the Pro404 (FIGS. 1A, 1C).

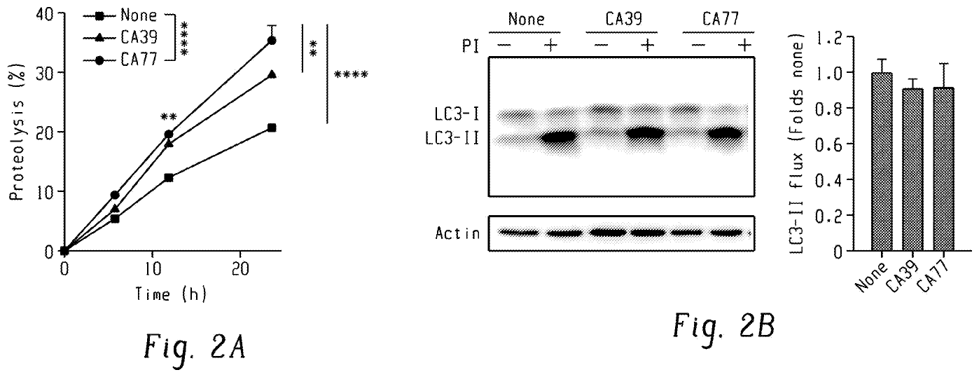

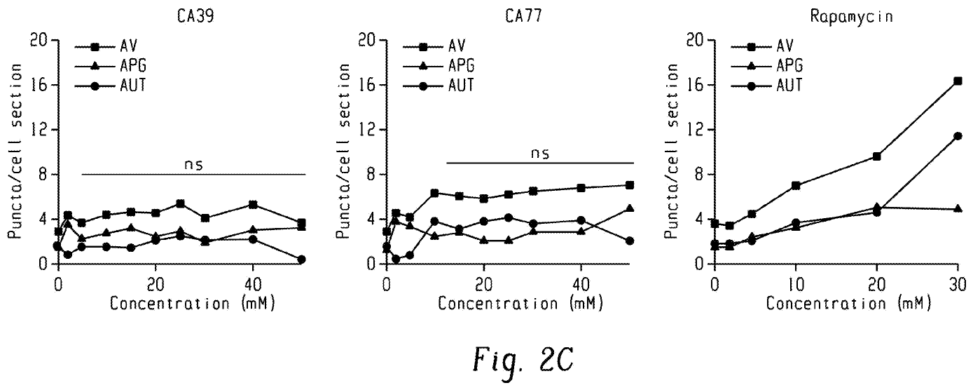

We confirmed that CA39 and CA77 activate CMA in a time and dose-dependent manner with higher potency than AR7 and that, in contrast to AR7, activation was still noticeable 12 h after washing out the compounds from the media (FIGS. 1D, 1E. Both compounds also efficiently activate CMA in other mouse cell types including neuronal-related cells and in human cells where the activating effect of AR7 was very discrete (data not shown). Interestingly, the activation of CMA elicited by CA77 in the neuron-related cells persisted and even further increased after the compound was removed from the media indicating a more robust and prolonged activating effect on these cells. CA39 and CA77 induced the expected increase in intracellular rates of degradation of long-lived proteins associated with CMA upregulation, but in contrast with AR7, for which the increase in protein degradation was sustained only for the first 12 h after addition of the compound, protein degradation remained upregulated 24 h after adding CA39 and CA77 (FIG. 2A) and did not have the inhibitory effect on macroautophagy, previously described for typical RARα antagonists (FIGS. 2B, 2C). After addition of any of the three compounds, we did not find significant differences in the percentage of protein degradation dependent on macroautophagy (sensitive to the macroautophagy inhibitor MRT, the rate of lysosomal degradation of LC3-II, or the number of autophagosomes and autolysosomes detected using the fluorescent tandem reporter mCherry-GFP-LC3. Thus, CA39 and CA77 are more potent CMA activators than the AR7 while still preserving their selectivity for this autophagic pathway.

### Example 2. CA Selectively Modulate a Subset of the RARα Transcriptional Program

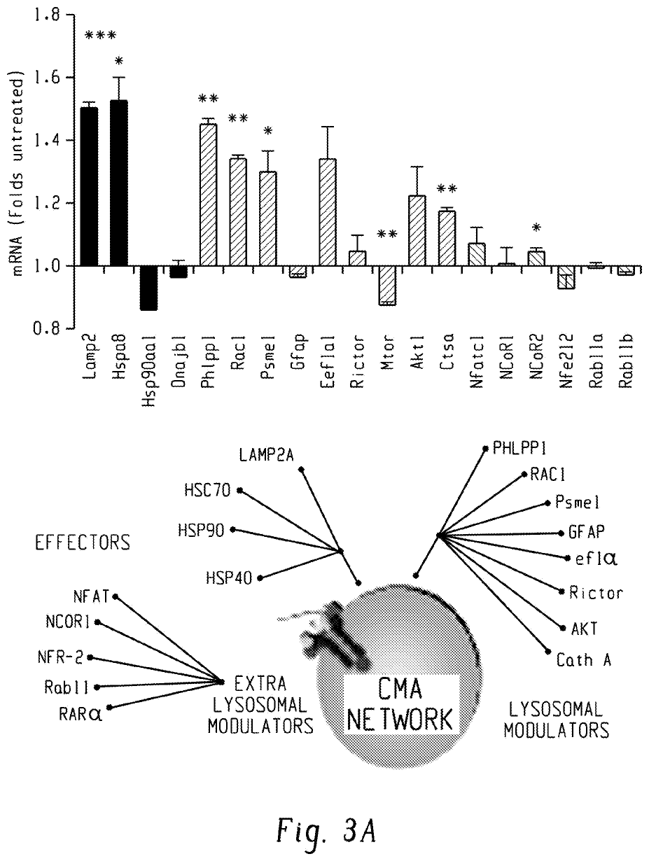

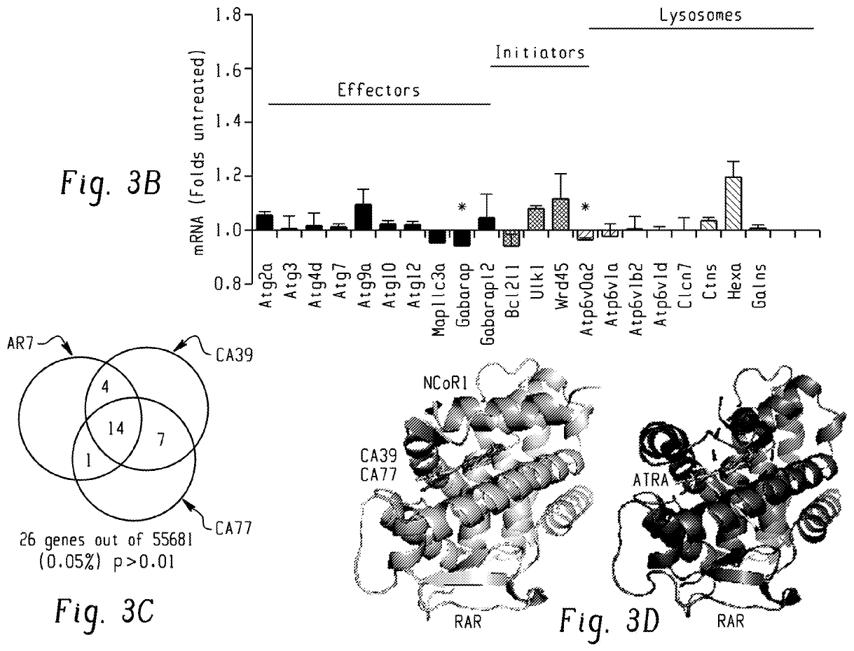

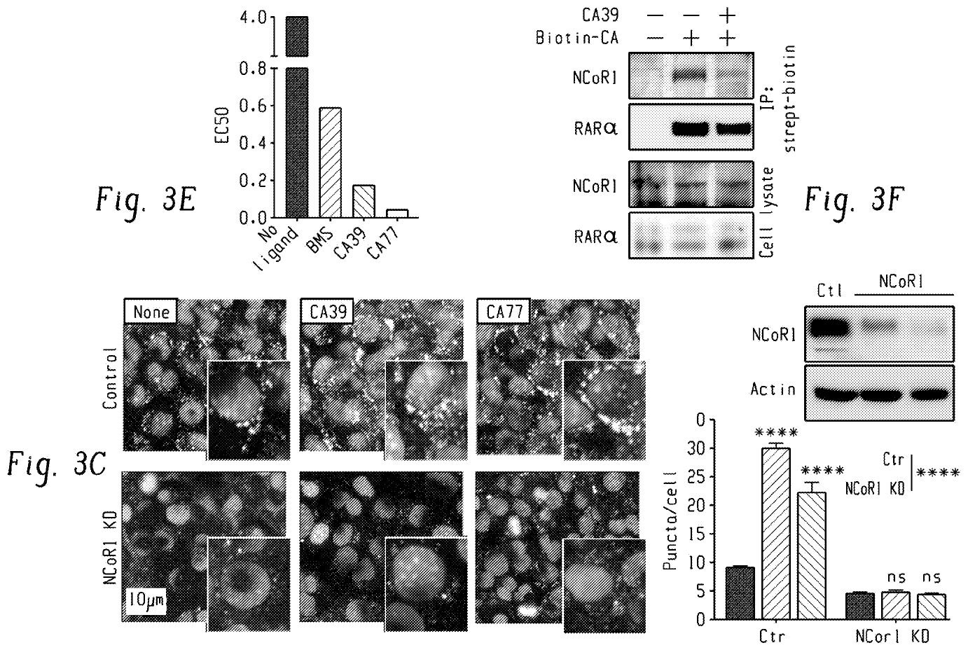

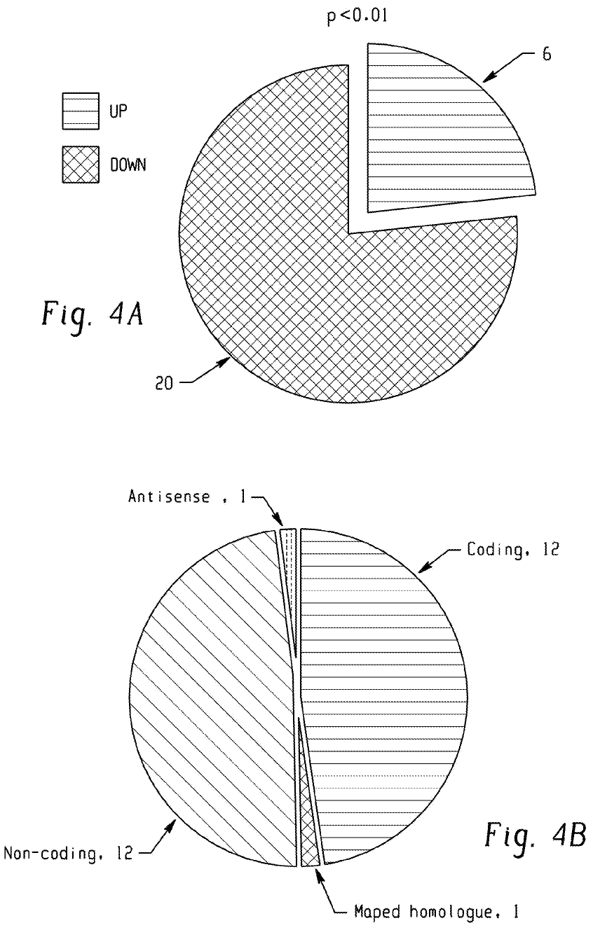

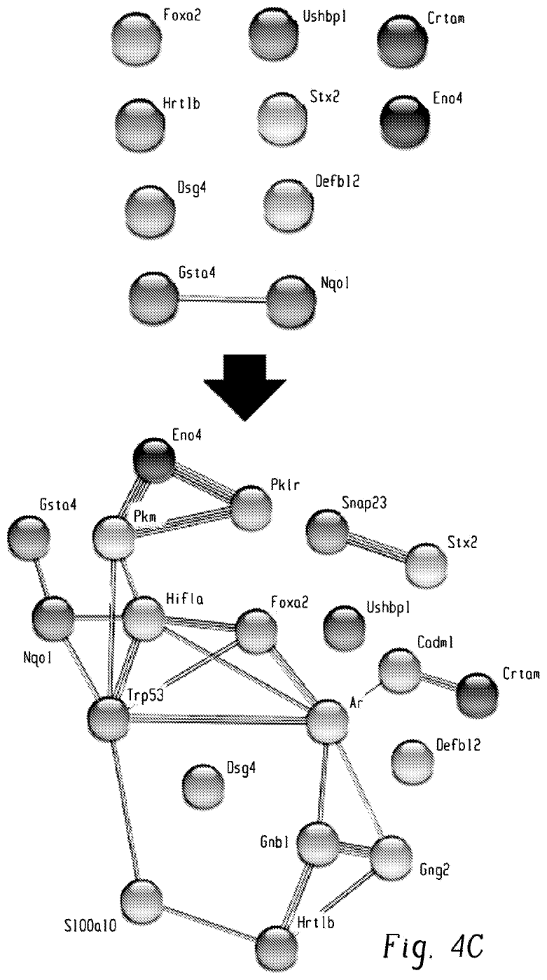

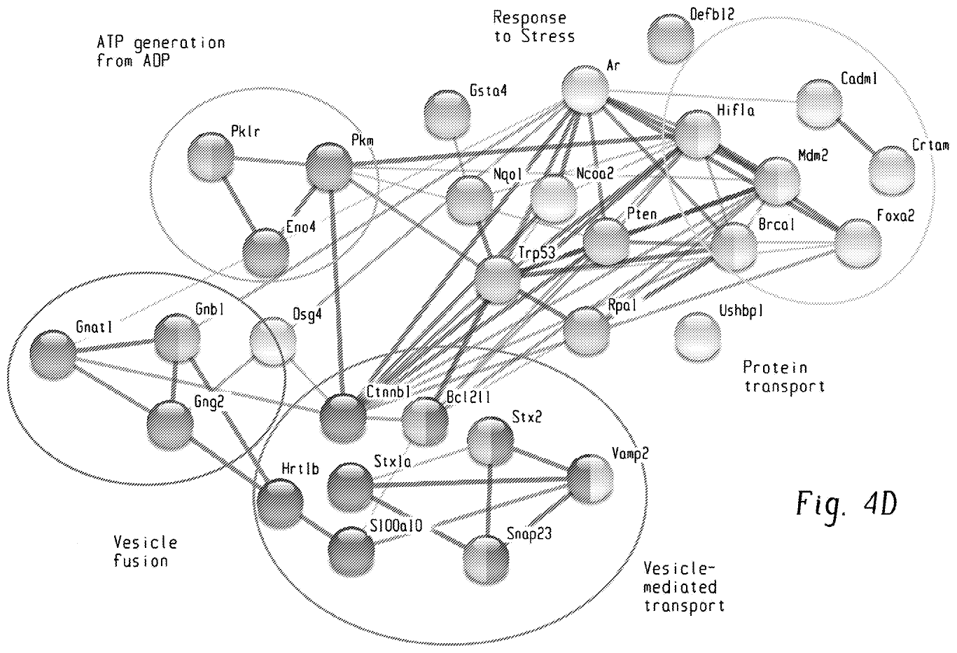

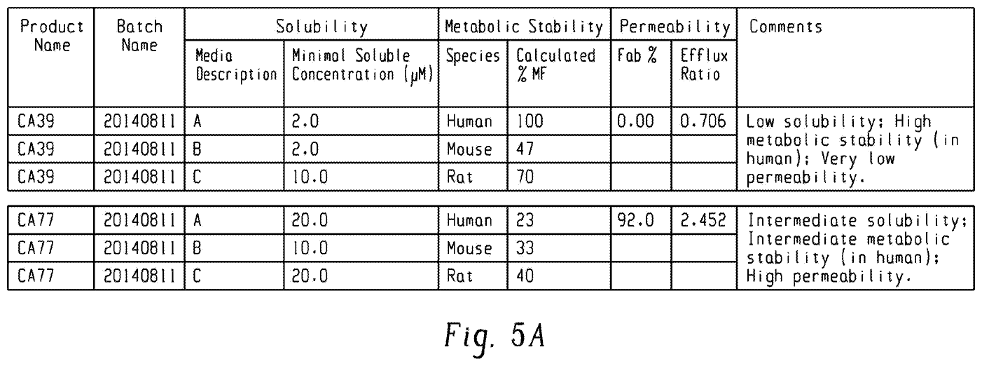

To elucidate the mechanism and basis for the selectivity of activation of CMA by AR7, CA39 and CA77 molecules, we performed comparative transcriptomic analysis of cells treated with BMS614, a selective RARα antagonist or with the original AR7 molecule (data not shown). In contrast with the marked changes in the transcriptome of cells treated with BMS614, AR7 treatment only induced changes in the expression of a discrete fraction of genes, including 8 of the 17 currently accepted components of the CMA network (FIG. 3A). (Kircher, P., et al., PLOS Biol. (2019) 18: e3000301) Quantitative qPCR for the 17 CMA components confirmed changes in expression upon addition of the CA compounds, manifested as both upregulation of CMA effectors and reduced expression of CMA inhibitors. This analysis also demonstrated differences in the magnitude of the changes in gene expression between AR7 and the new CA (CA39 and CA77) that could explain the higher CMA activation capacity described for the latter in the previous section. In fact, applicants have recently demonstrated that differences in CMA activity can be inferred by analyzing the expression of the subset of genes that participate in CMA and calculating a CMA activation score (by adding weight and directionality to each of the components in the CMA network). Using the observed CA-induced transcriptional changes, we found that the predicted CMA score was higher for CA39 and CA77 when compared to AR7. We did not find significant upregulation in the expression of effectors and regulators of macroautophagy and the lysosomal system (CLEAR network; selected genes shown in FIG. 3B). Similar analysis with CA39 and CA77 (FIG. 3C and FIGS. 4A and 4B), confirmed that beyond the 8 CMA-related genes, only 26 additional genes (11 coding and the rest non-coding or antisense) were modulated by the compounds (14 for all three compounds, and 12 for two and changed in the same direction with the third compound). Some of these proteins and non-coding RNAs could be yet unknown CMA effectors/regulators, as gene set enrichment and node expansion analysis (using STRING database) placed them on pathways that could interact with CMA such as G-protein coupled signaling, vesicular fusion, intermediate filaments organization and ATP generation, and all of them are involved in the cellular response to stress (FIGS. 4C and 4D).

Overall, our data supports that, as predicted, CA molecules affect only a very specific subset of RARα regulated genes in which the components of the CMA network are highly represented. These findings highlight major differences between conventional RARα antagonists and the CA compounds and points toward a very different mechanism of action.

### Example 3. CA Stabilize Binding of NCoR1 and RARα

The discrete transcriptional impact of CA compounds and their ability to stabilize RARα in an inactive conformation made us to consider that they may enhance interaction of co-repressor molecules with RARα such as NCoR1. A selective effect of the CA compounds on the co-repressor/receptor interaction could thus explain changes in only a very small subset of RARα-regulated genes. Docking to the NCoR1 bound RARα crystal structure, suggested that compounds' binding pose is compatible with NCoR1 binding to RARα but incompatible with the active RARα conformation that cannot bind NCoR1 or the CA compounds due to steric clashes (FIGS. 3D,1A). Indeed, CA39 and CA77 enhanced NCoR1 peptide binding to RARα in fluorescence polarization assays (FIG. 3E) and cellular RARα and NCoR1 could be co-immunoprecipitated after interaction with a biotinylated CA compound shown below (FIG. 3F), consistent with the docking and binding data. Knock-down of NCoR1 significantly decreased CMA activity and completely ablated the activating effect of CA39 and CA77 on CMA (FIG. 3G), thus supporting that the effect of the CA compounds is NCoR1-dependent.

We conclude that selectivity of CA compounds for CMA stems from their ability to stabilize RARα interaction with co-repressor NCoR1 and therefore preventing RARα adopting its active conformation, which requires binding of ATRA substrate and recruitment of co-activators.

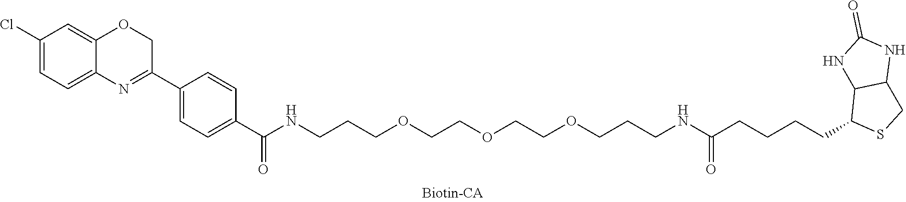 [SMILES: O=C(CCCC[C@H]1SCC2NC(=O)NC21)NCCCOCCOCCOCCCNC(=O)c1ccc(C2=Nc3ccc(Cl)cc3OC2)cc1]

### Example 4. CA Compounds Activate CMA In Vivo

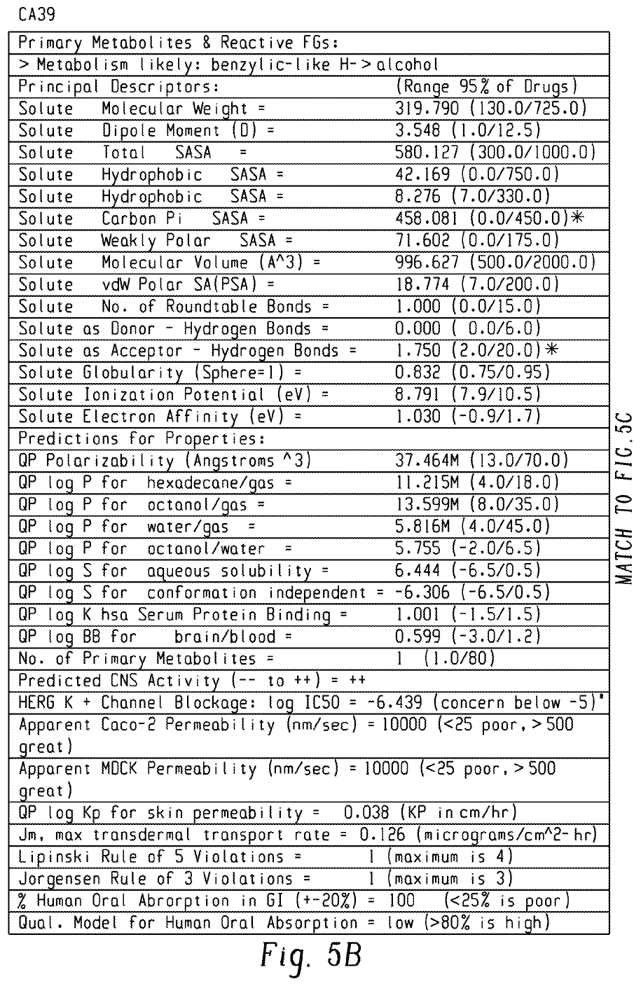

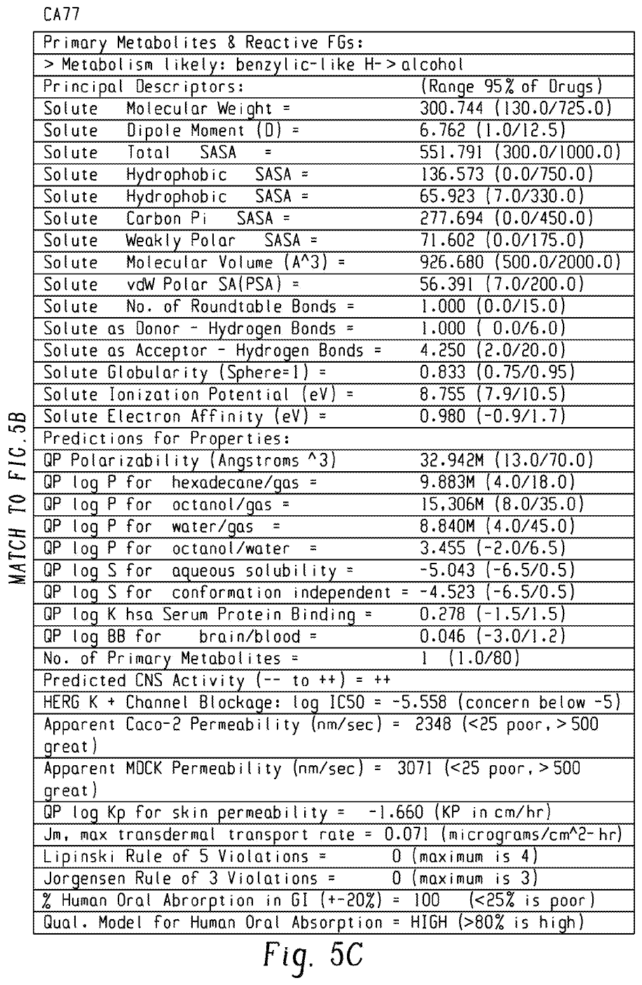

CA39 and CA77 have drug-like properties with reasonable solubility, high to intermediate metabolic stability with human liver microsomes and CA77 is also highly membrane permeable (FIG. 5A). Qikprop analysis using ADME properties prediction scores both CAs positive for CNS activity, oral bioavailability, permeability, and unlikely to show HERG K+ channel blocking activities (FIG. 5B).

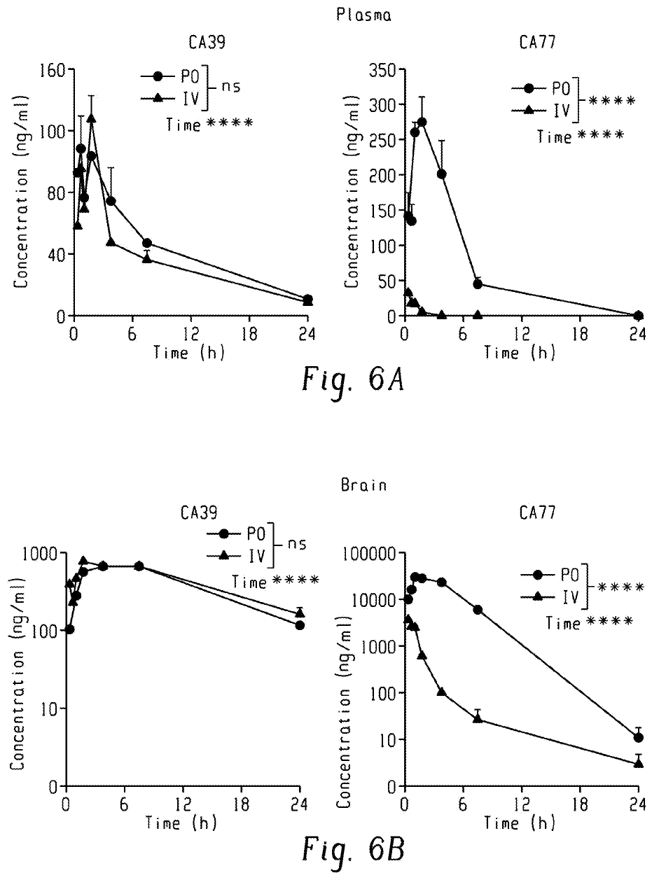

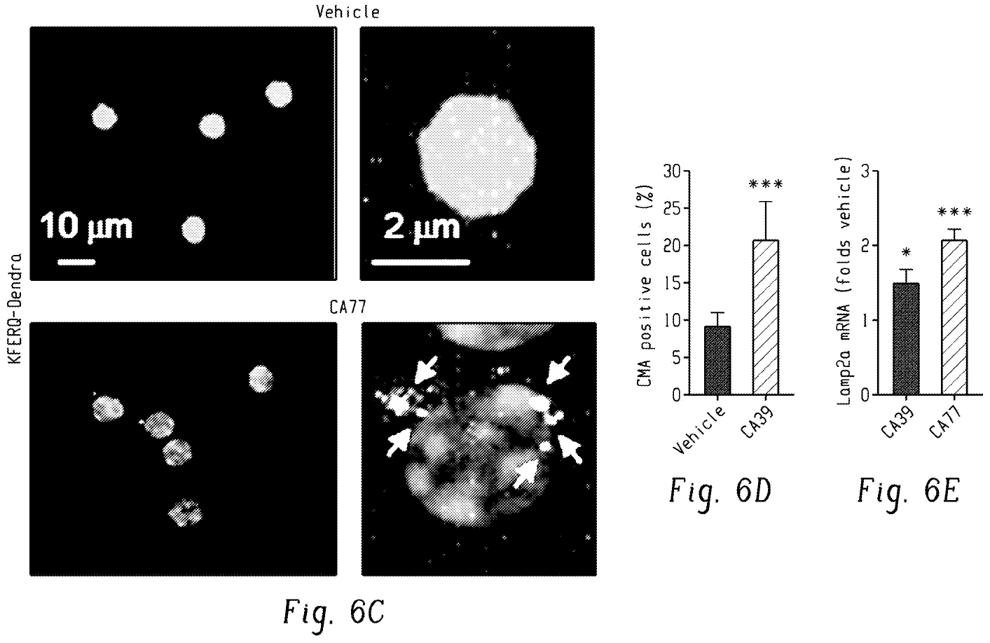

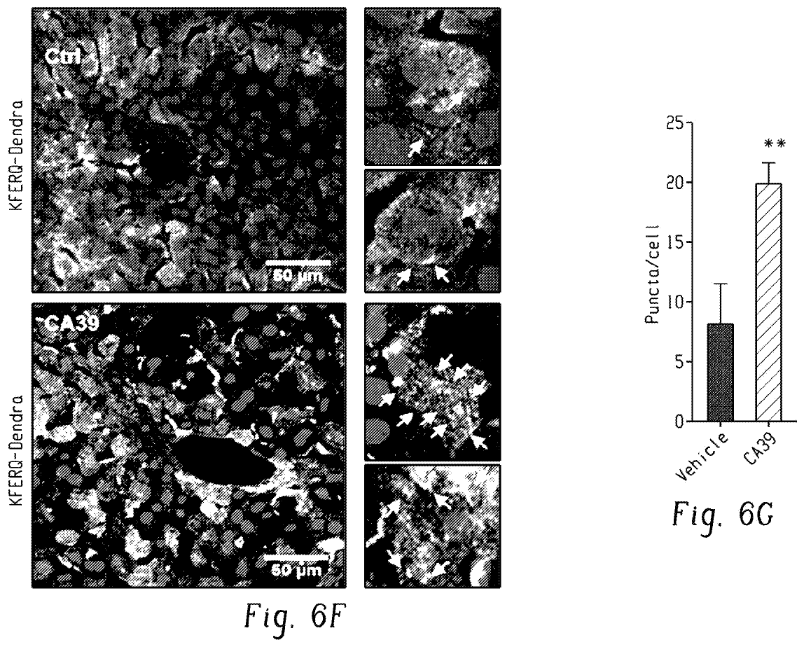

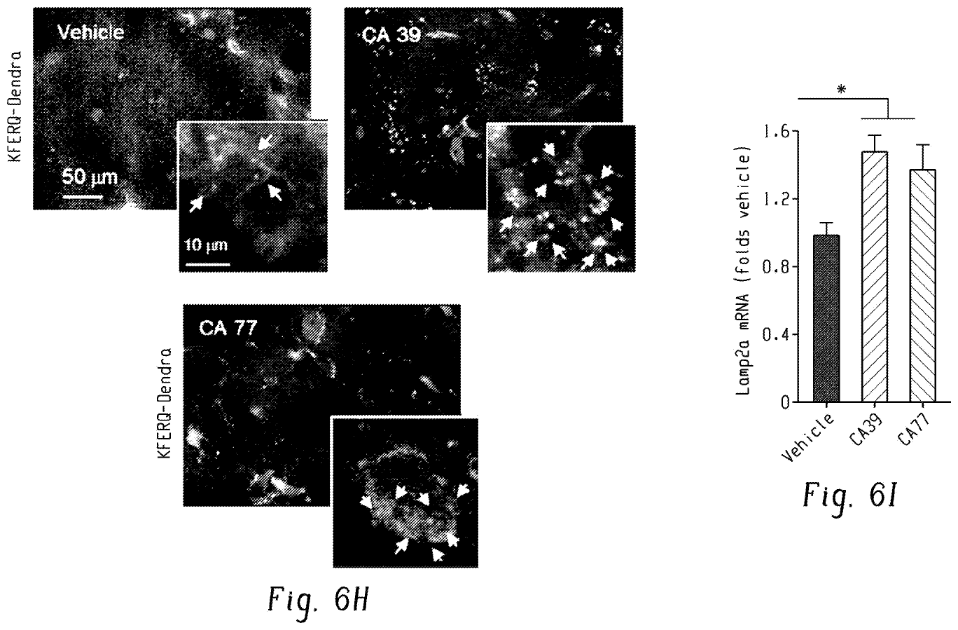

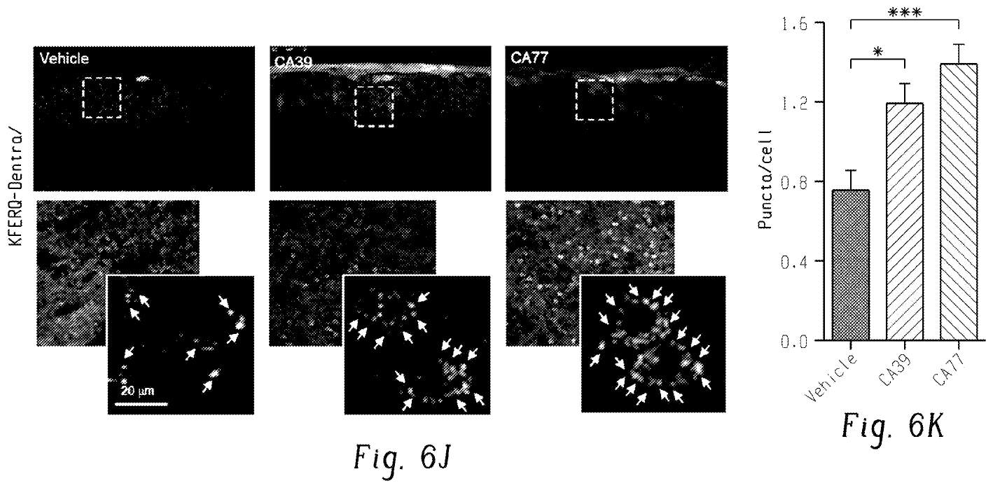

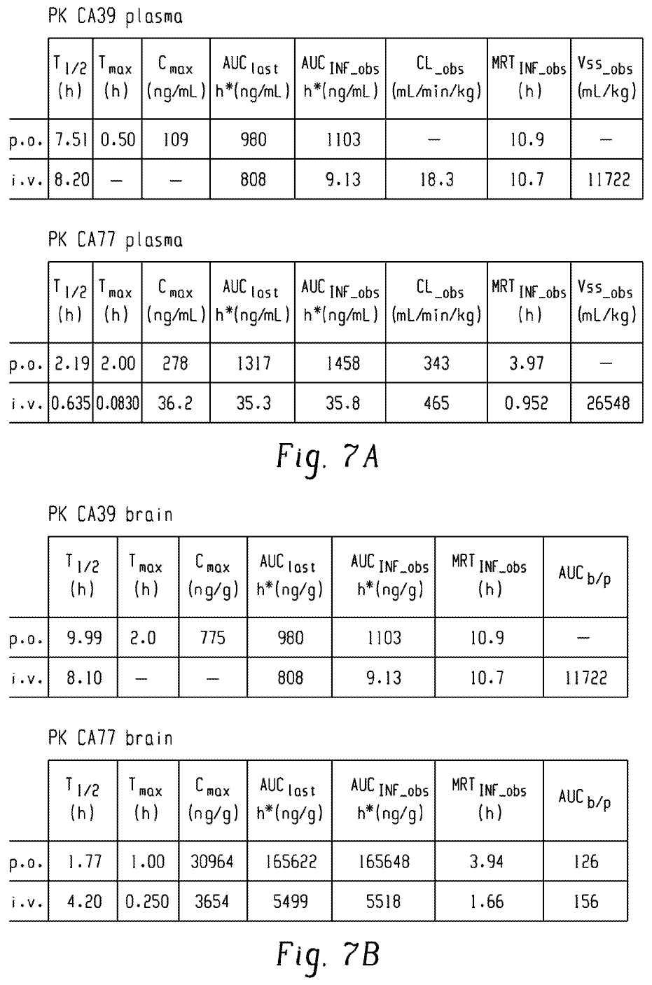

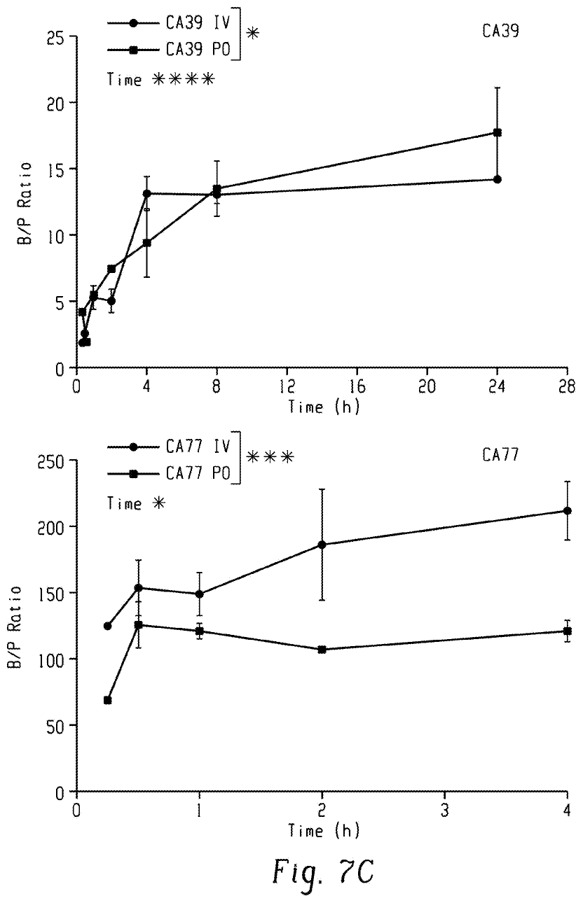

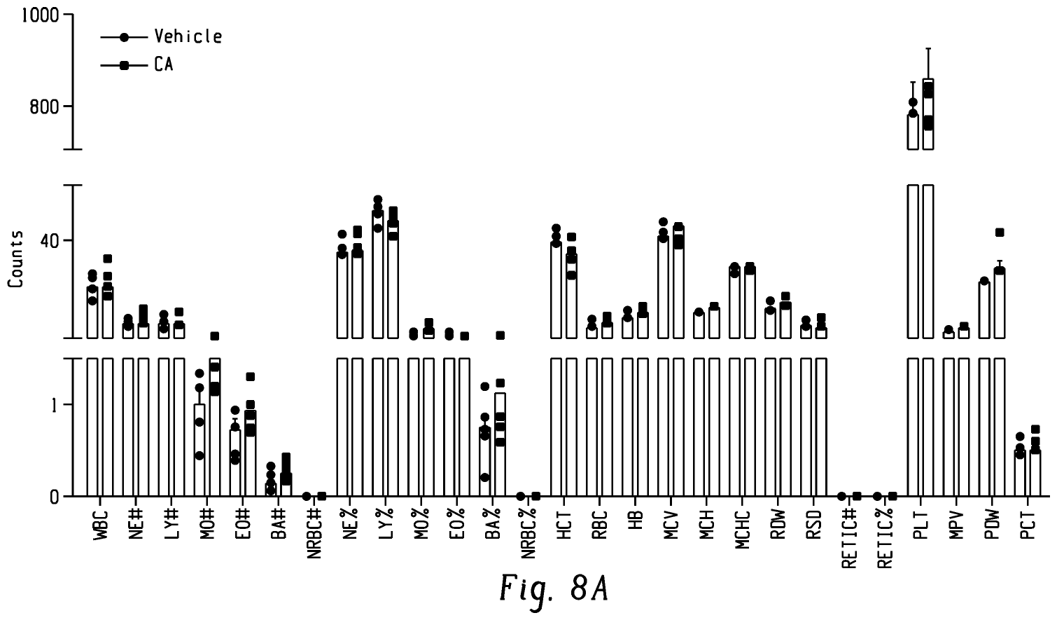

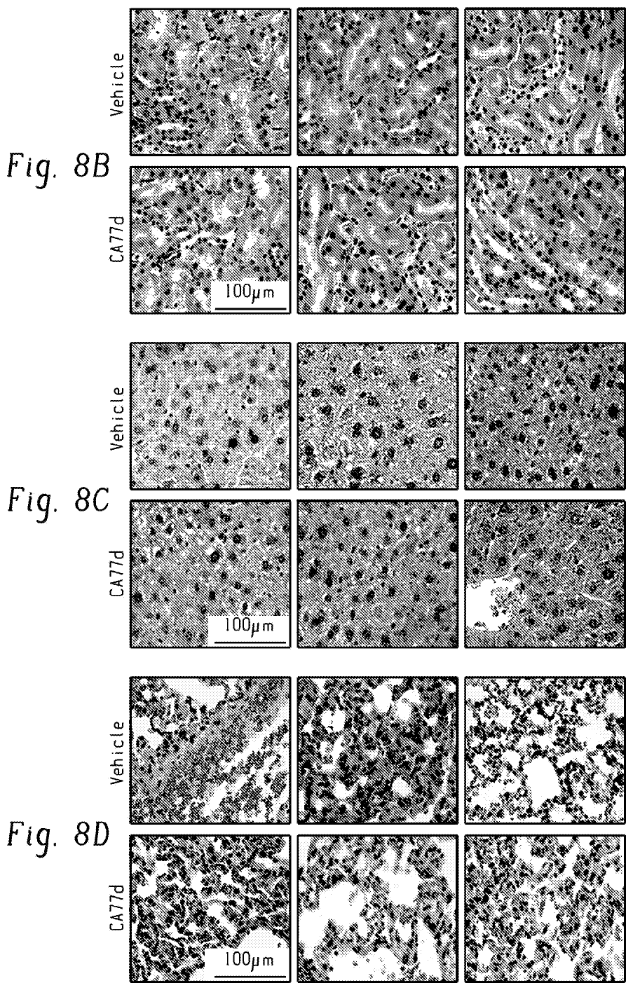

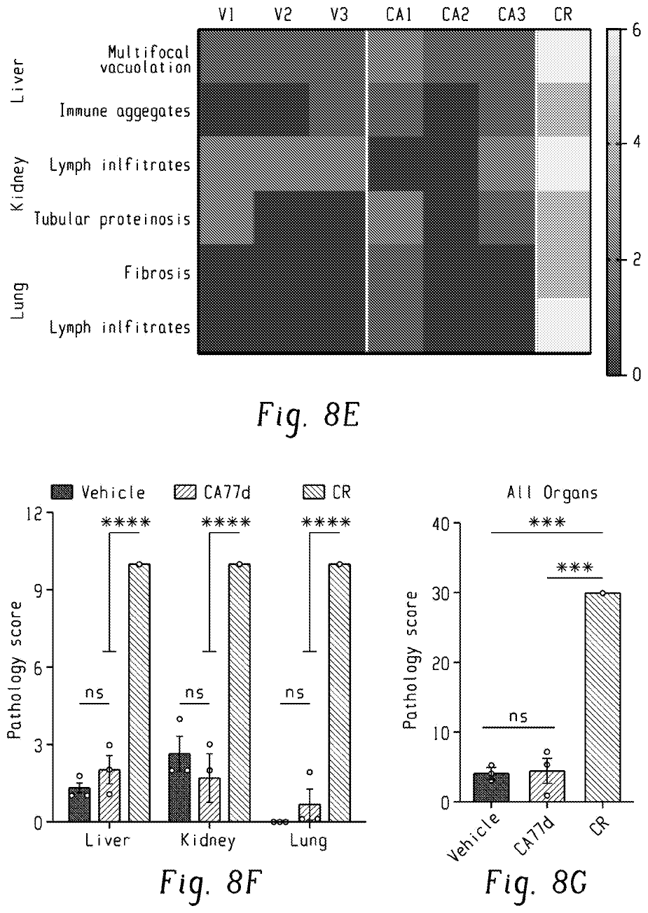

In vivo pharmacokinetics (PK) studies by intravenous (IV) administration of 1 mg/kg and oral administration (PO) of 30 mg/kg, showed that CA39 and CA77 have good biodistribution when administered intravenously or orally, with half-lives in plasma when administered by PO were 7.5 and 2.2 hr for CA39 and CA77, respectively and 8.2 h and 0.6 h for CA39 and CA77, respectively (FIG. 6A and FIG. 7A). Both molecules efficiently crossed the brain blood barrier and displayed significant brain exposure particularly for CA77, with half-lives in brain of 8 h by IV and 10 hr by PO for CA39 and 1.8 hr by IV and 4.2 hr by PO for CA77 (FIG. 6B and FIG. 7B). Their high brain to plasma ratio (FIG. 7C) and lack of peripheral blood or major organ (liver, lung, kidney) toxicity in mice upon chronic (5 months) daily oral administration of a more stable CA77 derivative (CA77.1; Plasma half-life 3 hr. and AUC brain/plasma 5.73 when administered orally) (FIG. 8), support that these compounds are suitable lead compounds for targeting central nervous system (CNS) chronic diseases.

Using a transgenic mouse model with systemic expression of the CMA reporter (KFERQ-Dendra mice) that allows visualizing CMA activity as a change in fluorescence distribution from a diffuse cytosolic pattern to fluorescent puncta, we next demonstrated the ability of CA39 and CA77 to activate CMA with comparable potency in vivo both in peripheral blood cells and in multiple organs (FIG. 6C-6K). CMA upregulation and increasing L2A mRNA levels were detected in isolated CD4+ T cells of the treated mice, providing a convenient method to test for target engagement during administration of CMA activating drugs (FIGS. 6C-6E). Both CMA activity and L2AmRNA levels also significantly increased in liver and midbrain upon administration of the CA molecules (FIG. 6F-6J). Systemic administration of CAs was also effective in upregulating CMA in the retina, with remarkable activation of this pathway in the cells of the outer nuclear layer of the retina (rods and cones) (FIG. 6F, 6H).

### Example 5. CA have a Cyto-Protective Effect Against Oxidative Stress

CMA is upregulated in response to oxidative stress and has been shown to be protective against this insult both in vitro and in vivo. The beneficial effect of activation of CMA under these conditions is a combination of its ability to selectively eliminate oxidized proteins through lysosomal degradation and of adjusting the cellular metabolic activity to reduced free radical production. We used oxidative stress paradigms in cultured cells and tissue explants to test the cytoprotective effect of the CA compounds in these settings.

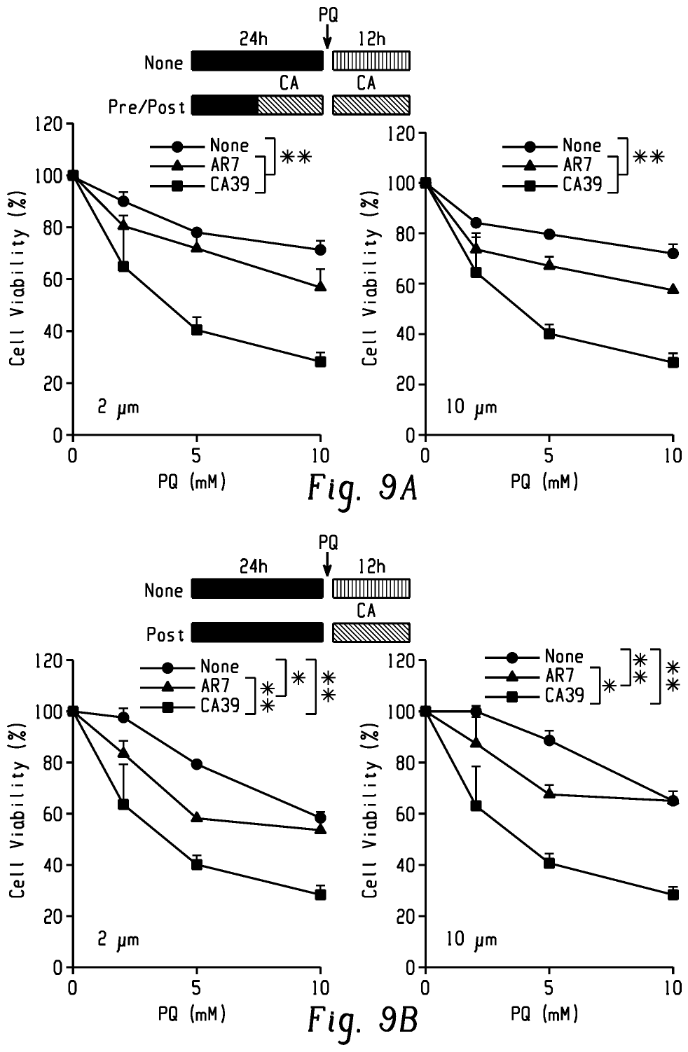

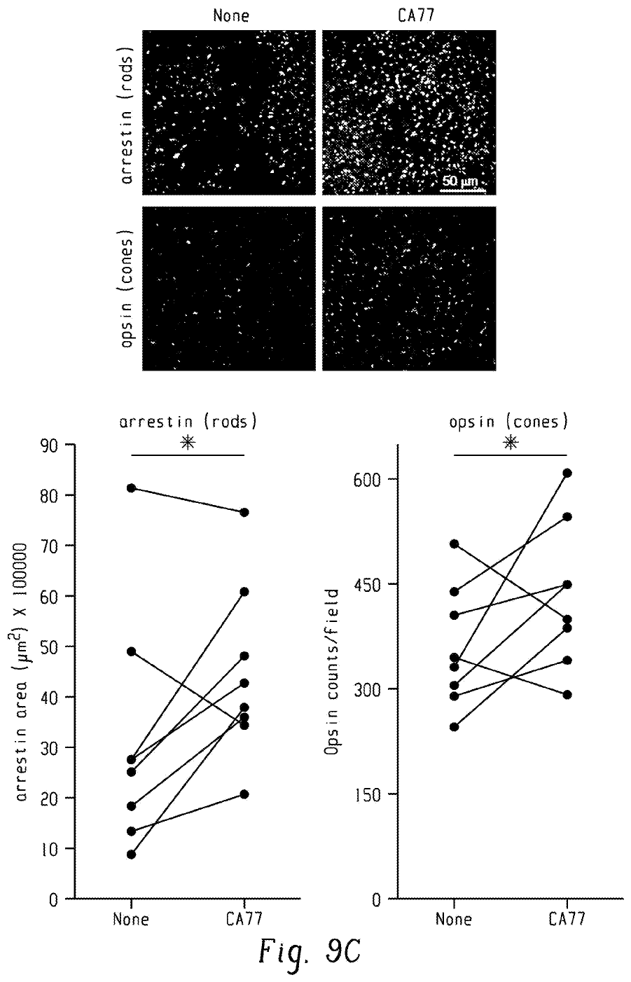

We exposed cultured cells to increasing concentrations of the pro-oxidant agent paraquat (PQ) and confirmed that CA39 reduced cell death when administered before the insult (FIG. 9A), as previously shown also for AR7, but it displayed significantly higher protection than AR7 when added after the insult (FIG. 9B).

To test if the protective effect of CA was also noticeable in whole tissues, we used CA77 in retinal explants of rd10 mice, an experimental model of retinitis pigmentosa. Rd10 mice harbor a missense mutation in the Pde6b gene and display photoreceptor cell death and vision loss similar to the disease progression in humans. Upregulation of L2A—but not of components of other autophagy pathways—has led to propose that CMA may be upregulated as part of the retinal response to prolonged starvation. Administration of CA77 to whole retinal explants led to significant preservation of the number of rods (rod arrestin-positive) and higher preservation also for cones (opsin-positive) when compared to the contralateral untreated retinal explant (FIG. 9C).

These results support improved cytoprotective effect of the new CMA activators and demonstrate their ability to function in whole tissues.

### Example 6. Systemic Administration of CA Ameliorates Retinal Degeneration

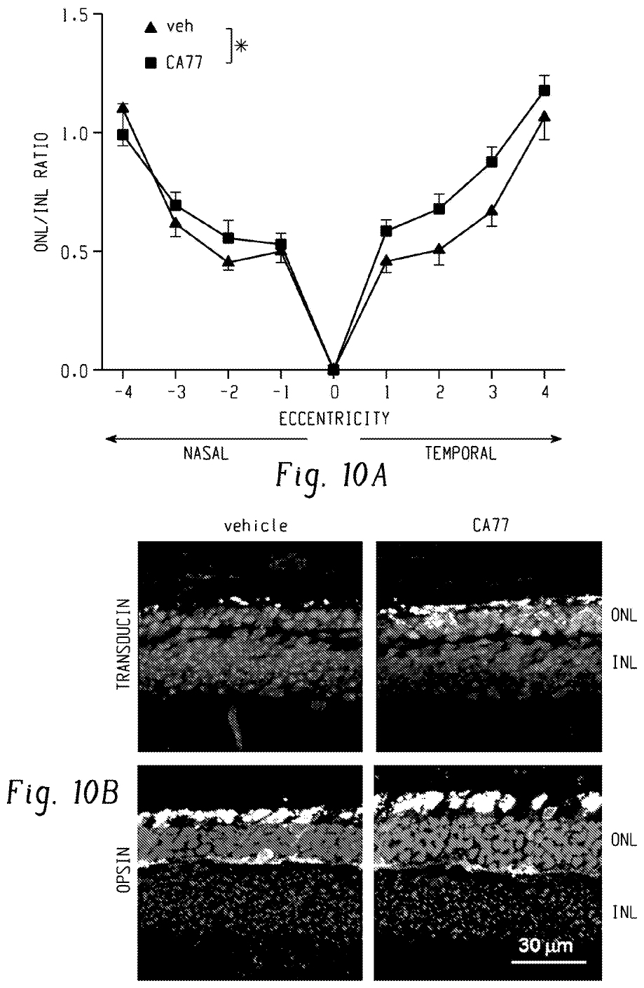

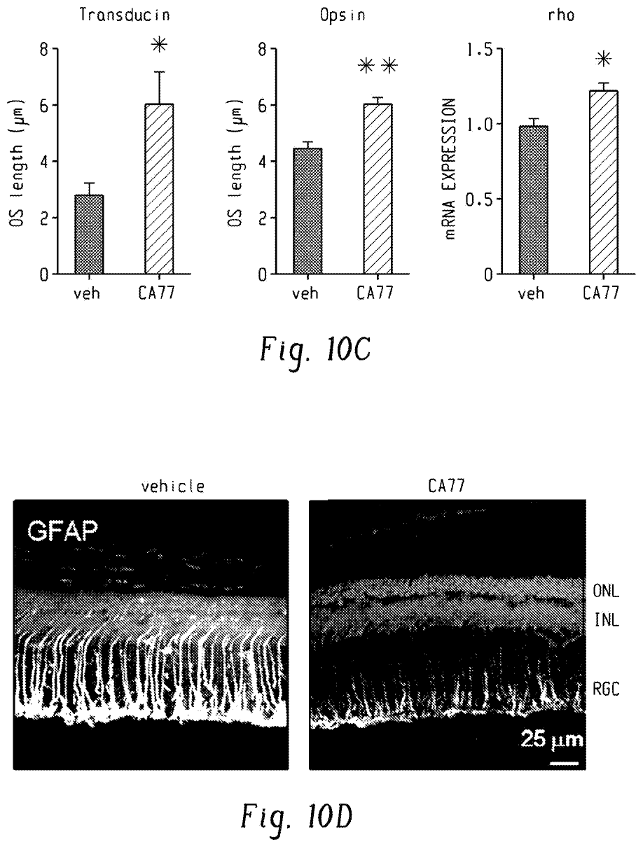

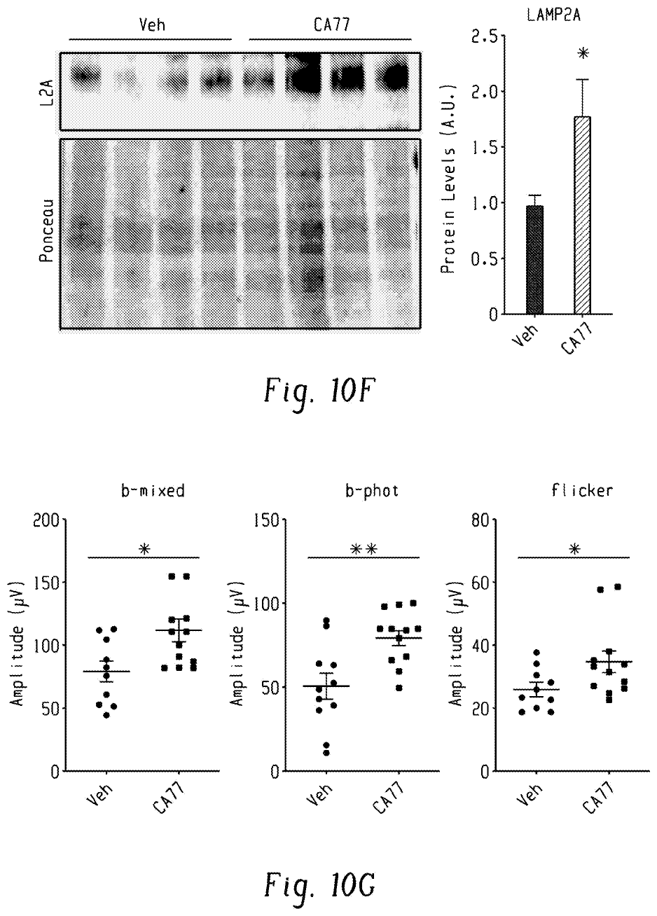

The favorable in vivo PK properties of CA39 and CA77 (FIG. 6A, 6B and Extended Data FIG. 6), their ability to activate CMA in whole animals (FIG. 6) and their remarkable protective effect in the rd10 retinal explants (FIG. 9C) motivated us to evaluate their therapeutic potential in the retinitis pigmentosa model in vivo (FIG. 10). In this model, photoreceptor loss starts with acute rod cell death that peaks at p22 and it is followed by a more progressive cone cell death²⁰. To make the intervention clinically relevant, we initiated the treatment around the peak of maximal rod death (from p18 to p25). We found that retinas from rd10 mice receiving daily intraperitoneal injection of CA77 (40 mg/kg bw) for one week showed significantly thicker outer nuclear layers (ONL), corresponding to photoreceptor nuclei, indicative of less cell loss (FIG. 10A).

Immunostaining with markers of rods (transducin) and red and green cones (opsin R/G) also revealed increased length of the outer segments of photoreceptors in the CA77-treated mice compared with those receiving vehicle (FIG. 10B, 10C). Higher mRNA levels of photoreceptor-specific proteins (i.e. rhodopsin shown in FIG. 10D) in these animals, further confirmed higher cellular preservation. We also compared levels of retinal inflammation as an additional marker of disease progression. As shown in FIG. 10E, retinas from CA77-treated mice have strikingly lower levels of GFAP, a well-known marker of astrocyte and activated Müller cells. Immunoblot for L2A demonstrate that the drug was effective in significantly increasing retinal levels of the limiting CMA component, thus confirming target engagement (FIG. 10F).

To confirm that the CA77-mediated improvement in retinal structure associated with preserved visual function, we performed electroretinograms (ERG). Mice receiving CA77 from P18 at P33 displayed significantly higher amplitude of their b-mixed, b-phot and flicker waves (FIG. 10G), which corroborate the beneficial effect of the intervention on retinal function. No significant differences in other light responses measured were observed between mice receiving CA77 or not.

We next evaluated a more translational approach by delivering the CA compound intravitreally, the preferred delivery rout in clinical practice. Analysis of rd10 mice retinas 7 days after a single intravitreal injection of CA77 (40 μM) at P18 revealed thicker retinal ONL/INL ratio and better OS and ONL preservation, both in cryosections and plastic embedded sections in drug-treated compared to vehicle-treated mice. Photoreceptor staining with rod and cone arrestin further corroborated the cytoprotective effect of CA77 in rd10 mice retinas. Gliosis was also significantly improved after CA77 administration preserved vision in the rd10 animal determined 7 days after the single intravitreal injection.

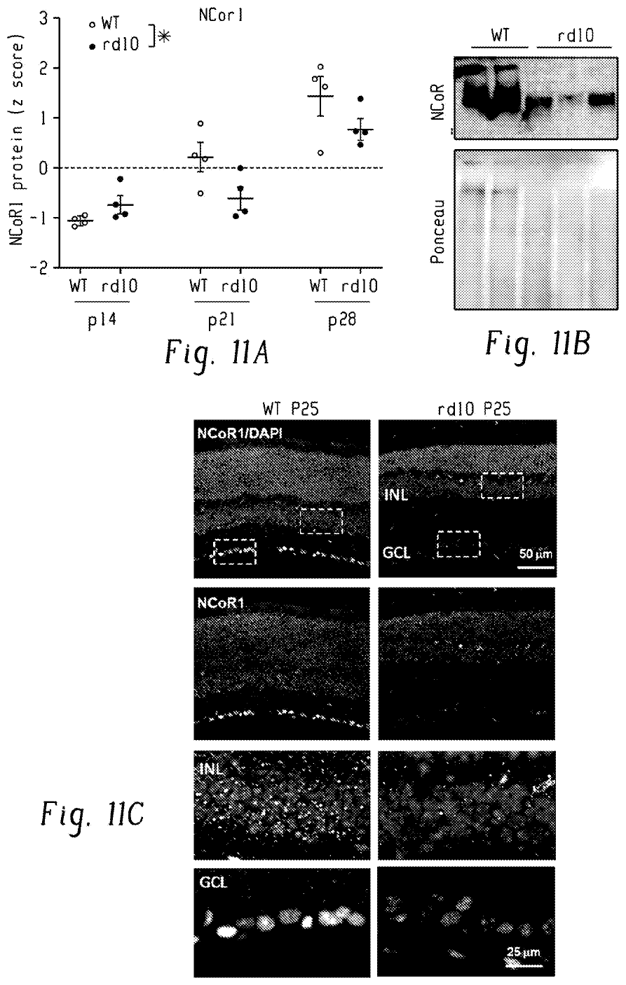

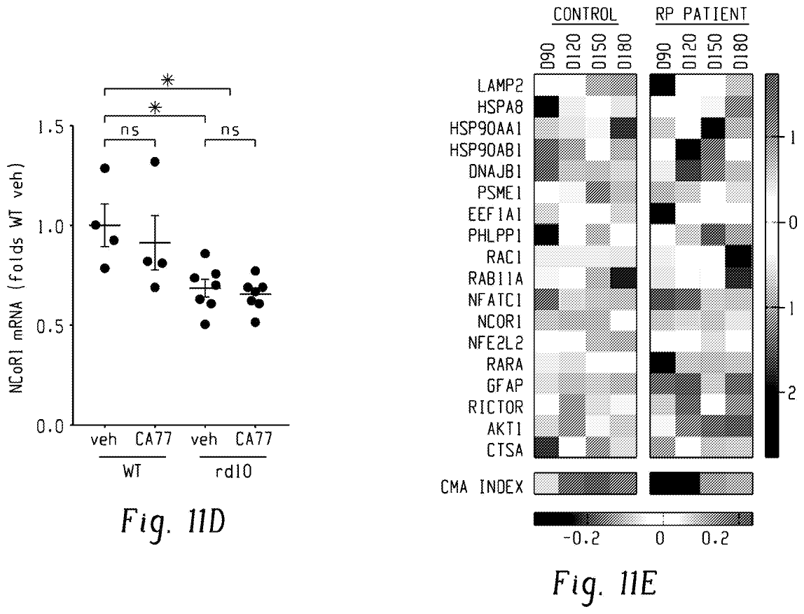

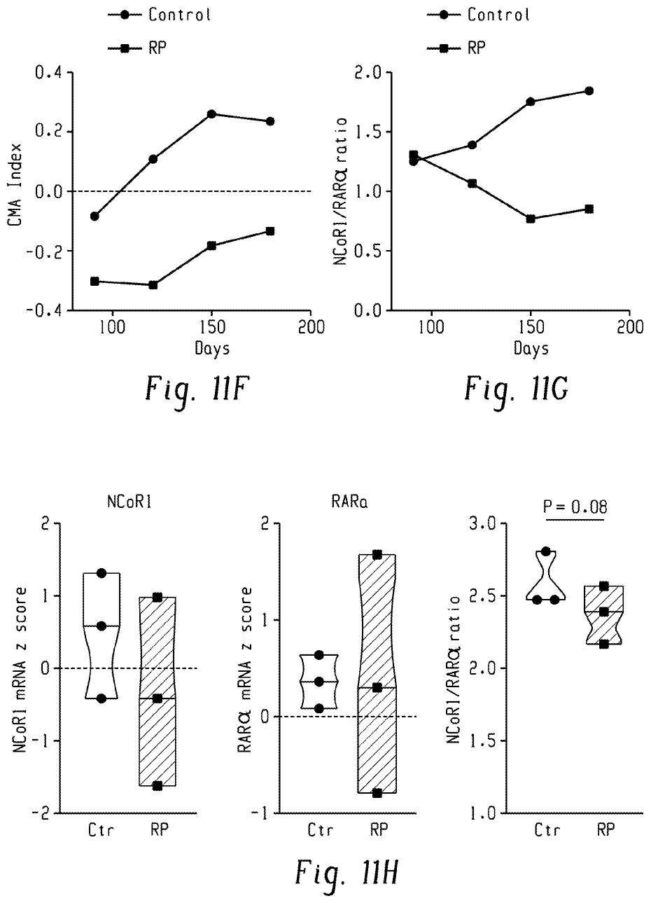

Interestingly, analysis of data from a proteomic study of retinas from rd10 mice at pre-, peak- and post-degeneration time points revealed a significant decrease in retinal levels of NCoR1 (FIG. 11A). We also confirmed by immunoblot and immunostaining for NCoR1 a very pronounced decrease of this protein in rd10 retinas compared to control retinas (FIG. 11B, 11C). Reduced levels of NCoR1 in rd10 retinas seem to be primary a result of transcriptional down regulation of this gene (FIG. 11D). As expected from the mechanism of action of CA77 described in this work, the drug did not change the expression levels of NCoR1 in either of the experimental groups, further supporting that that CAs are able to restore CMA activity by stabilizing NCoR1/RARα interaction and thus maximizing the effect of the remaining NCoR1 protein.

Lastly, to investigate the possible translational value of CAs in the human disease, we analyzed the status of CMA-related genes and NCoR1 expression using data from a previous study in patient-specific retinal organoids shown to recapitulate RP features. Although direct measurement of CMA activity is not possible in human retina, differences in CMA activity can be inferred by analyzing the expression of the subset of genes that participate in CMA and calculating a CMA activation score (by adding weight and directionality to each of the components in the CMA network). We found that the CMA activation index increases with human retinal organoid maturation (considered to be fully developed by 150 days), whereas retinal organoids from RP patients with the PDE6B mutation (the same mutation as the rd10 mouse model) displayed a marked reduction in the CMA activation index at all stages (FIG. 11E, 11F). A pronounced increase in the ratio of co-repressor to receptor (NCoR1 to RARα ratio) weighted heavily in the observed upregulation of CMA in the early stages of maturation in healthy human retinal organoids (FIG. 11E, 11G, black line). In contrast, reduced NCoR1/RARα expression ratio, more noticeable as the retinal organoids reached full maturation, was observed in the RP patient retinal organoids (FIG. 11E, 11G, green line). We observed a similar trend toward reduced NCoR1 expression and increased RARα and overall reduced NCoR1/RARα expression ratio upon analysis of a second study using human iPSC-derived retinal organoids from RP patients bearing instead RP2 mutations, that account for approximately 15% of all cases of X-linked RP (FIG. 11H). These findings support that reduced NCoR1/RARα expression ratio and the subsequent lower CMA activity may be a common feature in RP patients and that interventions that stabilize the interaction of the co-repressor with the receptor, as the one described in this work, could be successful to restore the normal NCoR1/RARα tone in the human disease.

1. A method of stabilizing the interaction of a Retinoic Acid Receptor-alpha (RARα) and a corepressor, Nuclear Receptor Corepressor 1 (NCoR1) comprising contacting the RARα with an amount of a Chaperone Mediated Autophagy (CMA) Activator sufficient to stabilize the RARα-NCoR1 interaction.

2. The method of claim 1, wherein the RARα is contacted with the CMA Activator in vivo in a subject identified as having a risk factor for a neurodegenerative disorder.

3. (canceled)

4. A method of upregulating CMA gene expression in a subject comprising administering a sufficient amount of an CMA activator to upregulate the expression of at least one effector or activator gene associated with CMA.

5. The method of claim 4, wherein the gene expression of at least one effector gene selected from LAMP2A, HSC70, HSP90AA1, HSP90AB1, HSP40, EEF1A1, PHLPP1, and RAC1 is increased in the subject relative to the expression of the effector gene in the patient prior to administration of the CMA activator or the expression level of at least one activator gene selected from NFATC1, NCOR1, NFE2L2, NFR-2, RARα, and Rab11 is increased in the subject relative to the expression of the activator gene in the patient prior to administration of the CMA activator.

6. A method of preventing or slowing the advancement of a neurodegenerative disorder in a subject having an early symptom or biomarker of the neurodegenerative disorder, comprising administering an amount of a CMA activator sufficient to stabilize the interaction of RARα and the corepressor NCoR1 in vivo.

7. The method of claim 6, wherein the biomarker is beta-amyloid or tau and the method further comprises determining the progression of beta-amyloid and/or tau pathology by positron emission tomography (PET) and/or magnetic resonance (MR) imaging.

8. The method of claim 2, wherein the neurodegenerative disorder is Alzheimer's disease (AD), Lewy body dementia, Parkinson's disease (PD), Huntington's disease, Amyotrophic lateral sclerosis (ALS), Frontotemporal dementia (FTD), Spinocerebellar ataxias (SCAs), and Progressive subcortical gliosis.

9. The method of claim 2, wherein the neurodegenerative disorder is AD, and the subject does not suffer from dementia.

10. The method of claim 6, wherein the neurodegenerative disease is AD, and the early symptom is decreased cognitive function, anxiety, disinhibition, movement disorders, memory loss and/or confusion, difficulty concentrating, difficulty completing daily tasks, time and/or place confusion, difficulty with visual images and/or spatial relationships, difficulty conversing, misplacing objects, poor judgment, withdrawal from activities, olfactory dysfunction, changes in mood and personality.

11. The method of claim 6, wherein the neurodegenerative disease is AD, and the biomarker is tau protein (total tau or phosphorylated tau) or beta-amyloid (e.g., Aβ42) in the plasma or cerebrospinal fluid (CSF) of the subject.

12. A method of maintaining preventing or slowing the advancement of a retinal degenerative disorder or preserving vision in a subject having an early symptom or biomarker of the retinal degenerative disorder, comprising administering to the subject an amount of a CMA activator sufficient to stabilize the interaction of RARα and the corepressor NCoR1 in the subject's retina.

13. The method of claim 12, wherein the retinal degenerative disorder is retinitis pigmentosa.

14. (canceled)

15. A method of increasing Lamp 2A levels in neurons or retina of a subject in need of treatment for an age-related neurodegenerative disorder or retinal degenerative disorder, comprising
administering to the subject an amount of a CMA activator sufficient to stabilize the interaction of RARα and the corepressor NCoR1 in the subject's retina or neurons.

16. The method of claim 15, wherein the CMA Activator is an Activator capable of hydrogen bonding with Thr 233 in the RARα.

17. The method of claim 15, wherein the CMA Activator is an Activator capable of hydrophobic interaction with at least one of the following RARα (human consensus sequence) amino acids: Pro 407, Leu 409, Ile410, Pro408, and Ile 236 and/or with at least one of the following RARα (human consensus sequence) amino acids: Leu 266, Ile270, Phe302, and Leu305.

18. The method of claim 15, wherein the amount of the CMA activator administered is 0.01 mg/kg to 100 mg/kg, 0.1 mg/kg to 50 mg/kg, 0.1 mg/kg to 20 mg/kg, 0.1 mg/kg to 10 mg/kg, 0.1 mg/kg to 100 mg/kg, 1 mg/kg to 100 mg/kg, or 10 mg/kg to 100 mg/kg daily.

19. The method of claim 18, wherein the amount of CMA activator is administered to the patient daily for at least 3 months, at least 6 months, at least 9 months, at least 12 months, or at least 18 months.

20. The method of claim 2, wherein the subject is a human patient.

21. The method of claim 2, wherein the CMA Activator is administered as an oral, intravenous, parenteral, intranasal, sublingual, buccal, or ophthalmic dosage form.
